(19) United States
(12) Patent Application Publication
(10) Pub. No.: US 2003/0069471 A1
(43) Pub. Date: Apr. 10, 2003

(54) Medical image observation apparatus or microscopic system and method of displaying medical images

(75) Inventors: Kazuhito Nakanishi, Tokyo (JP); Masaaki Ueda, Sagamihara-shi (JP); Masahiko Kinukawa, Huntington Station, NY (US)

Correspondence Address: Scully, Scott, Murphy & Presser, 400 Garden City Plaza, Garden City, NY, 11530, US

(73) Assignee: OLYMPUS OPTICAL CO., LTD., TOKYO (JP)

(21) Appl. No.: 10/241,215

(22) Filed: Sep. 11, 2002

(30) Foreign Application Priority Data
Sep. 11, 2001 (JP) 2001-275546

(51) Int. Cl. A61B 1/00

(52) U.S. Cl. 600/101

(57) ABSTRACT

A medical image display in which a positional relationship between images from different image sources can be easily understood is disclosed. The microscopic system displays a live image of an operation and a preoperative image having the same range in a switched manner. The system preferably has a microscope for magnified observation of an operated part, an endoscope for observing the operated part, an image display section for displaying a plurality of images: a preoperative image including the vicinity of the part under operation; a preoperative image having the same range as an observing range of the endoscope; and an image observed by the endoscope, a switching section for switching the images displayed in the image display section, and a display control section for controlling the order of switching according to an instruction for switching from the switching section.

### CROSS-REFERENCE TO RELATED APPLICATION

This application is based upon and claims the benefit of priority from the prior Japanese Patent Application No. 2001-275546, filed Sep. 11, 2001, the entire contents of which are incorporated herein by reference.

### BACKGROUND OF THE INVENTION

1. Field of the Invention

The present invention relates to a medical image observation apparatus and, more particularly, to a surgical microscope system.

2. Description of the Related Art

A surgical microscope is sometimes used in combination with an endoscope to carry out an operation accurately. In such cases, positions to be observed by the surgical microscope and the endoscope are often displayed on a preoperative image using a navigation system. During an operation as described above, the endoscope is used to allow enlarged observation of a part of an image observed with the surgical microscope or an operated part at a dead angle. In order to know correlation (orientation) between an image observed with a surgical microscope and an image observed with an endoscope, the operator must have an understanding of each of a positional relationship between the image observed with the surgical microscope and a navigation image (preoperative image) and a positional relationship between the image observed with the endoscope and the navigation image. Operators have understood a relationship between a position observed with an endoscope with a position observed with a surgical microscope by matching two positional relationships as described above in their minds.

According to the related art as described above, the understanding of a positional relationship between images has been a burden to operators.

The invention provides a system that allows an operator to have an understanding of a positional relationship between observed images easily.

### SUMMARY OF THE INVENTION

According to the invention, medical observation apparatus (e.g., a surgical microscope or endoscope) generates a preoperative image having a range that is substantially the same as a field of observation based on preoperative image data and displays the preoperative image and an observed image on a display section in a switching manner.

A microscope system will be described by way of example. In a microscope system having a microscope section for observing an operated part and a sub-mechanism for observation (e.g., an endoscope) for observing the operated part, a preoperative image having a, range that is substantially the same as the field of observation of the sub-mechanism for observation is generated using preoperative data (that is obtained using CT or MRI, for example). The preoperative image and an image obtained by the sub-mechanism for observation are displayed on the display section in a switching manner.

Since the microscope system is capable of displaying the image obtained by the sub-mechanism for observation and the preoperative image having substantially the same range while switching them quickly, an operator can compare the image obtained by the sub-mechanism for observation and the preoperative image easily.

Preferably, the microscope system also generates a preoperative image having a range greater than the field of observation of the sub-mechanism for observation, and the preoperative image having a wider range is selectively displayed on the display section along with the preoperative image having the same range and the image obtained by the sub-mechanism for observation. Since this allows an operator to refer to the preoperative image having a wider range too, the range of observation of the sub-mechanism for observation can be easily recognized.

Preferably, the microscope system displays the image on the display section in a part of the field of view of the microscope section. This allows an operator to observe the image displayed on the display section while observing a microscopic image (a live image) through an eyepiece portion of the microscope section.

Preferably, the microscope system has an image switching section for sequentially switching displayed images according to operations on an input section (e.g., a push switch) performed by an operator, and the image displayed on the display section is displayed through an operation on the image switching section. With such a configuration, since displayed images are switched according to operations of an operator, the operator can observe a desired image at desired timing. For example, an operator can repeatedly and alternately display an image obtained by the sub-mechanism for observation and a preoperative image having the same range a desired number of times at desired timing to compare those images.

It is also advantageous to input a desired position of the sub-mechanism for observation (e.g., an endoscope) to the system in advance and to identify correlation between an actual position of the sub-mechanism detected with an observing position detection mechanism and the desired position. This makes it possible to generate an image that indicates the correlation between the desired position and the actual position. An example of an image thus generated is an image of the sub-mechanism for observation overlapped with a contour of a target for the desired position or an arrow indicating a direction in which the sub-mechanism for observation is to move to reach the desired position.

### BRIEF DESCRIPTION OF THE DRAWINGS

These and other features, aspects, and advantages of the apparatus and methods of the present invention will be better understood with regard to the following description, appended claims, and accompanying drawings where:

FIG. 1 illustrates an internal configuration of a binocular eyepiece tube section of a surgical microscope;

FIG. 2 is a side view of the configuration in FIG. 1;

FIG. 3 illustrates a configuration of a lens body;

FIG. 4 is a perspective view of an LCD optical system;

FIG. 5A is a perspective view showing an internal configuration of an X-Y table;

FIG. 5B is a block diagram of a control system;

FIG. 6 is a perspective view showing a general system configuration of a surgical microscopic apparatus;

FIG. 7 illustrates an endoscope according to an embodiment of the invention;

FIG. 8 illustrates an image that is an preoperative image having an observing position of an endoscope for the preoperative image and a viewing direction of the endoscope superposed thereon;

FIG. 9 illustrates an image in which a stereoscopic preoperative image having the same range as the observing range of the endoscope is displayed and in which a viewing direction of the endoscope is superposed on an image observed by a microscope;

FIG. 10 illustrates display in which the image displayed in the field of view has been switched to an image observed by the endoscope;

FIG. 11 is a block diagram of a control system in a third embodiment of the invention;

FIG. 12 illustrates another example of an endoscope according to the invention;

FIG. 13 illustrates orders for switching of images displayed in an in-field display section in response to pressing first and second switches;

FIG. 14 is a side view of an alternative configuration of FIG. 1, according to a fifth embodiment of the invention;

FIG. 15 is a block diagram of a control system in the fifth embodiment of the invention;

FIGS. 16A, 16B, and 16C illustrate contents displayed in an in-field display section in the fifth embodiment of the invention;

FIG. 17 illustrates a display that appears when an image displayed in the in-field display section and an image displayed by a second observation optical system change place with each other;

FIG. 18 illustrates a flow of an image display caused by first and second switches;

FIG. 19 is a flow chart illustrating steps for setting display screens;

FIG. 20 illustrates a display image setting screen;

FIG. 21 is a block diagram showing a configuration of a sixth embodiment of the invention;

FIG. 22 illustrates a position desired by an operator that is displayed in a preoperative image;

FIG. 23 shows an observation image that is an image observed by an endoscope having a contour of the position desired by an operator in a preoperative image superposed thereon;

FIG. 24 illustrates how an endoscope is guided to an image of a desired position input by an operator in a preoperative image;

FIG. 25 is a block diagram showing a seventh embodiment of the invention;

FIG. 26 illustrates how an image mixer synthesizes an image observed by a surgical microscope and an in-field image into a surgical microscope image that is observed by an operator;

FIG. 27 is a diagram showing a configuration in which a W-VHS recorder is used instead of using two VTR's as in FIG. 25;

FIG. 28 illustrates contents displayed by an in-field display section in an eighth embodiment of the invention;

FIG. 29 illustrates contents displayed by the in-field display section and a second observation optical system in the eighth embodiment of the invention;

FIG. 30 illustrates contents displayed by the in-field display section and a second observation optical system in the eighth embodiment of the invention;

FIG. 31 illustrates contents displayed by the in-field display section when an ultrasonic probe is used in the eighth embodiment of the invention;

FIG. 32 illustrates contents displayed by the in-field display section and the second observation optical system when an ultrasonic probe is used in the eighth embodiment of the invention; and

FIG. 33 illustrates contents displayed by the in-field display section and the second observation optical system when an ultrasonic probe is used in the eighth embodiment of the invention.

### DETAILED DESCRIPTION OF THE INVENTION

Preferred embodiments of the present invention will be described hereinafter with reference to the drawings.

First, each of the preferred embodiments will be schematically described.

A first embodiment of the invention employs a surgical microscopic apparatus as a microscope system. The microscopic apparatus has a surgical stereoscopic microscope and an endoscope as sections for observing an operated part. A part of the field of view of the surgical microscope is allotted for a region for displaying a separate image, and various preoperative images and live images from the endoscope are displayed in the region. Images displayed in the field of view of the surgical microscope are switched in a sequence stored in advance according to operations on one switch provided on a side of the endoscope. An arrow indicating a viewing direction of the endoscope is superposed on an image observed by the surgical microscope.

A second embodiment of the invention is a modification of the first embodiment. In the second embodiment, two switches are provided on a side of an endoscope, and the order of switching images displayed in the field of view of a surgical microscope is reversed by operating the switches alternately.

A third embodiment of the invention is a modification of the second embodiment. In the third embodiment, images in the field of view of a surgical microscope are switched by identifying the images from identification signals from sources of the images (images are identified from the positions of connectors at which video signals are input in the first and second embodiments).

A fourth embodiment of the invention is a modification of the third embodiment. In the fourth embodiment, connectors having different shapes are assigned to respective images sources, and images and the connectors are associated with each other in an n versus 1 relationship. To switch images in the field of view of a surgical microscope, a switch on a side of an endoscope is operated, which causes an image selector to select the connectors in an order that is recorded in advance (the same connector may be consecutively selected a plurality of times).

A fifth embodiment of the invention is a modification of the third embodiment. In the fifth embodiment, a second observation optical system is provided in the vicinity of an eyepiece portion of a surgical microscope. The second observation optical system displays a separate image using a liquid crystal outside the field of observation of the surgical microscope. Two switches are provided on a side of an endoscope, and the display in the field of view of the surgical microscope is sequentially switched (the order can be changed) as in the first embodiment when an operator presses one of the switches. When the operator presses the other of the switches, switching occurs between the display in the field of view of the surgical microscope and the display on the second observation optical system.

A sixth embodiment of the invention is a modification of the third embodiment. In the sixth embodiment, an operator inputs a position to be observed in a surgical microscope in advance. A contour of the desired position is superposed on a preoperative image displayed in the field of view of the microscope. An arrow indicating the direction of the desired position is superposed on an image observed by the surgical microscope.

A seventh embodiment of the invention discloses a technique for recording an image observed according to the fifth embodiment. In the fifth embodiment, there are two image outputs, i.e., an image output including an image observed by the surgical microscope and an image in the field of view of the surgical microscope, and an image output from the second observation optical system. There is disclosed a control section for synchronizing operations of two separate recording apparatus that record the two images, respectively. Alternatively, the two image outputs are synthesized into one screen that is recorded with a single recording apparatus.

An eighth embodiment of the invention is a modification of the fifth embodiment. In the eighth embodiment, one of the images displayed in the field of view of a surgical microscope is a preoperative image that is taken in the same direction as the observing direction of the surgical microscope (the position of an endoscope is displayed in the image). The position of the endoscope is displayed in the field of observation of the surgical microscope in alignment with the position of the endoscope in the image in the field of view. An example of a surgical microscopic apparatus is also described in which an ultrasonic probe is used in place of an endoscope.

The preferred embodiments will now be described in detail with reference to the drawings.

A surgical microscopic apparatus of the first embodiment has a surgical stereoscopic microscope and an endoscope as sections for observing an operated part. A small region for displaying separate images is accommodated in a part of the field of view of the surgical microscope (an image displayed in the field of view). Various preoperative images and live images from the endoscope are displayed in this region. The images displayed in the field of view of the surgical microscope are switched in an order that is stored in advance through operations on a switch provided on a side of the endoscope. An arrow indicating the viewing direction of the endoscope is preferably superposed on an image observed by the surgical microscope itself.

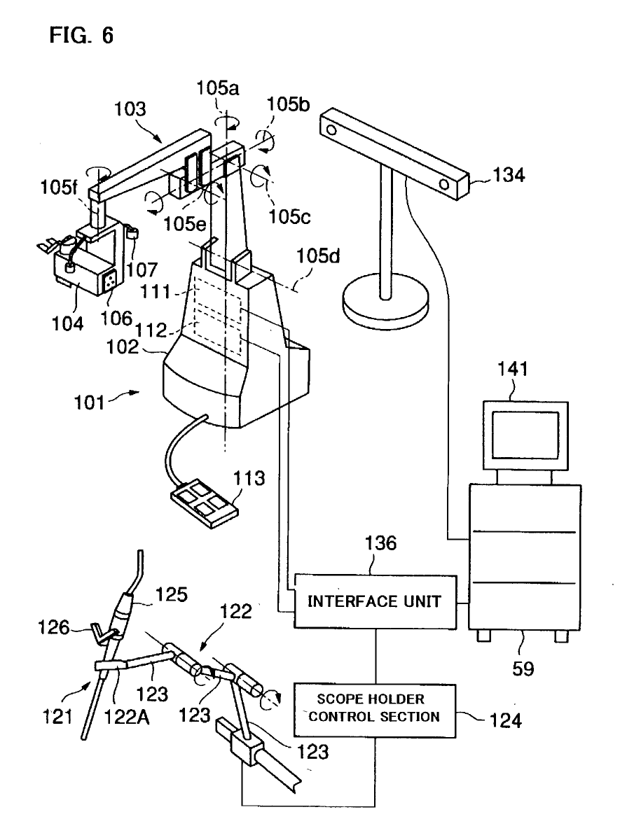

FIG. 6 shows the surgical microscopic apparatus as a whole. Major features of the same will be described. The surgical microscopic apparatus has a surgical microscope 101 having a lens body 104 and an endoscope 121 supported by a scope holder 122. There is also provided a digitizer 134 for detecting the positions of the lens body 104 of the surgical microscope and the endoscope 121. Those devices are connected to a navigation apparatus 59.

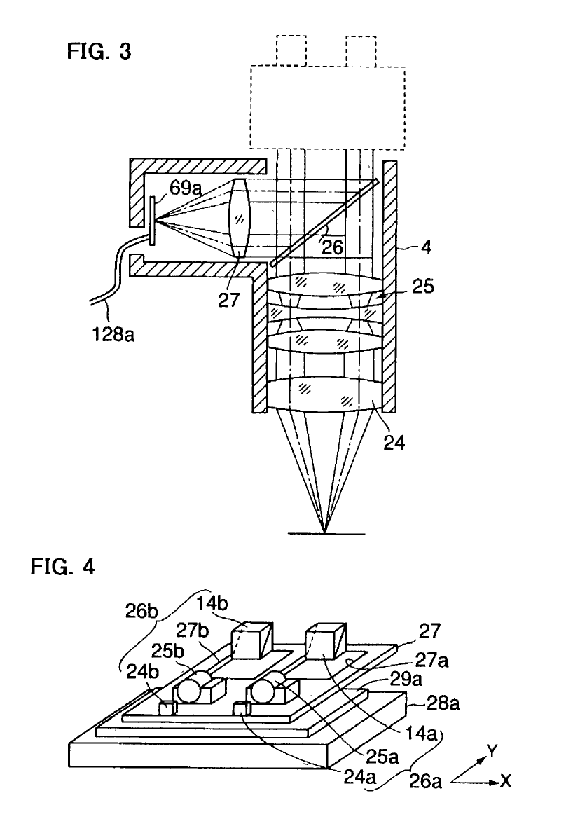

FIG. 3 shows a central part of the lens body 104 of the surgical microscope. A beam of light from an operated part is guided through an objective optical system 24 and a variable power optical system 25 to a binocular eyepiece tube section that is indicated by a broken line. An optical system 27 and a half mirror 26 superimpose an image of a monitor 69a on an observed image.

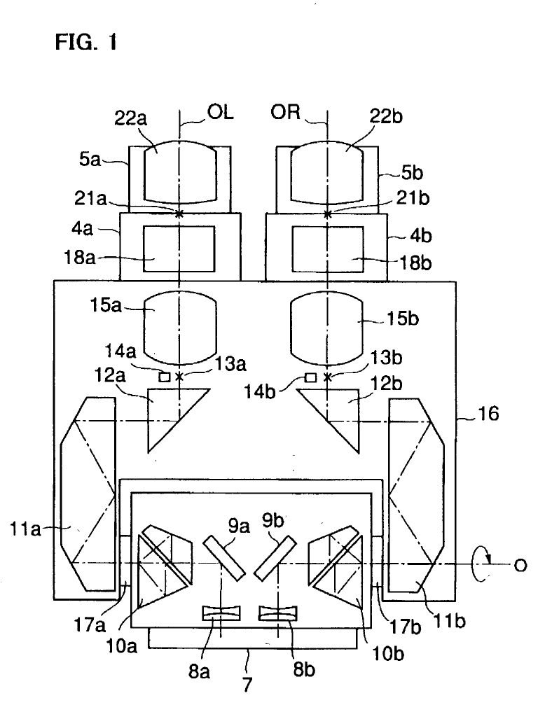

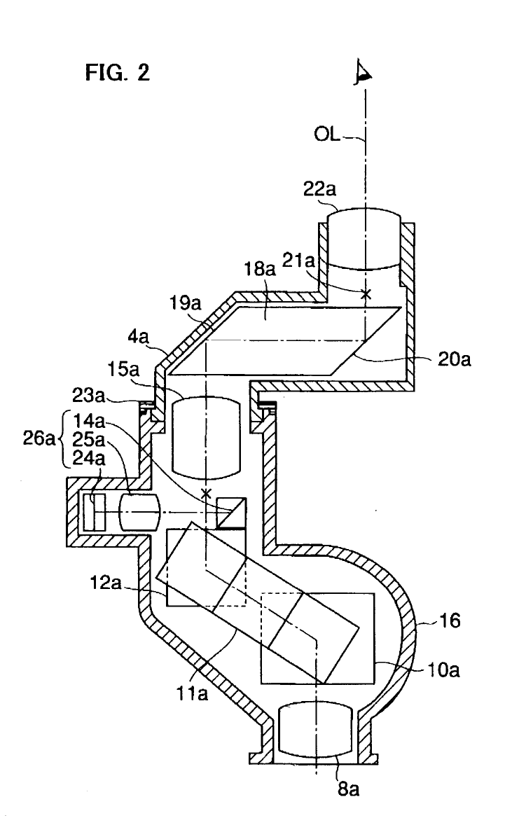

The binocular eyepiece tube section is shown in FIGS. 1 and 2. A beam of light from the configuration shown in FIG. 3 enters image forming lenses 8a and 8b and exits eyepiece optical systems 22a and 22b to be guided to the eyes of an operator. Prisms 14a and 14b located in the vicinity of intermediate image forming points 13a and 13b allocate a part of the field of an image observed by the surgical microscope for display of separate images (in-field images).

FIG. 4 shows a mechanism for displaying such in-field images. The prisms 14a and 14b are placed in parts of holes 27a and 27b through which the beam of light from the surgical microscope passes. Images of monitors 24a and 24b are formed on top surfaces of the prisms 14a and 14b by image forming lenses 25a and 25b.

An image 145 in FIG. 8 represents how the in-field display is presented in an image observed by the surgical microscope. An arrow 146 in FIG. 8 is superimposed on the image observed by the microscope (by the monitor 69a, the optical system 27, and the half mirror 26 in FIG. 3).

Each part will be described in detail with reference to FIGS. 1 to 10. FIGS. 1 and 2 show a configuration of optical systems of the ocular eyepiece tubes of the surgical microscope. FIG. 1 illustrates an internal configuration of the binocular eyepiece tube section. FIG. 2 is a side view showing a left observation optical system. The right observation optical system of the binocular eyepiece tube section is substantially similar to the left observation optical system illustrated in FIG. 2.

As shown in FIGS. 1 and 2, the binocular eyepiece tube section has a fixed housing 7, a movable housing 16 rotatably mounted to the fixed housing 7 through an axis O, a pair of interpupillary adjustment housings 4a and 4b rotatably mounted to the movable housing 16 about optical axes OL and OR as axes of rotation, and ocular housings 5a and 5b mounted to the interpupillary adjustment housings 4a and 4b, respectively.

A pair of left and right image forming lenses 8a and 8b are provided in the fixed housing 7. The image forming lenses 8a and 8b are optically connected with an observation optical system of a lens body (reference number 4 in FIG. 3), and left and right beams of observation light which are emitted from the minor body enter the same.

Mirrors 9a and 9b respectively reflect the beams of light that have passed through the image forming lenses 8a and 8b outwardly at angle of 90 deg. Image rotator prisms 10a and 10b are provided on exit optical axes of the mirrors 9a and 9b. Prisms 11a and 11b for inverting the two beams of observation light at an angle of 180 deg respectively are provided behind the image rotator prisms 10a and 10b. Further, triangular prisms 12a and 12b for reflecting the exit light from the prisms 11a and 11b in a direction in parallel with the observation optical axes OL and OR of the eyepiece optical systems to be, described later are fixed behind the same. First intermediate image forming points 13a and 13b formed by the image forming lenses 8a and 8b are located behind the triangular prisms 12a and 12b.

Prisms 14a and 14b as light guide sections are provided in the vicinity of the first intermediate image forming points 13a and 13b such that their top surfaces are substantially aligned. Relay lenses 15a and 15b for relaying images are fixed behind the first intermediate image forming points 13a and 13b. The prisms 11a and 11b, the triangular prisms 12a and 12b, and the relay lenses 15a and 15b are incorporated in the movable housing 16.

The movable housing 16 can rotate about the axis O (entrance optical axes of the prisms 11a and 11b) through connecting sections 17a and 17b. The rotator prisms 10a and 10b are rotated about the axis O by a cam mechanism (not shown) at an angle that is half the angle of rotation of the movable housing 16 relative to the fixed housing 7.

A parallel prism 18a has an entrance reflecting surface 19a and an exit reflecting surface 20a, and it is incorporated in the interpupillary adjustment housing 4a. An image relayed by the relay lens 15a from the first intermediate image forming point 13a exits the exit reflecting surface 20a of the parallel prism 18a to be formed at a second intermediate image forming point 21a. The image is guided to an eyepiece optical system 22a incorporated in the ocular housing 5a. The optical path constitutes the observation optical axis OL of the surgical microscope.

The interpupillary adjustment housing 4a is supported by the movable housing 16 such that it can be rotated about an axis that substantially agrees with the axis of light exiting the triangular prism 12a (the vertical direction of the figure). As shown in FIG. 2, the interpupillary adjustment housing 4a is prevented from moving in the axial direction by a stopper member 23a. This structure along with the parallel prism 18a forms a so-called Siedentopf-type interpupillary adjustment structure.

Similarly, a parallel prism 18b has an entrance reflecting surface and an exit reflecting surface, and it is incorporated in the interpupillary adjustment housing 4b. An image relayed by the relay lens 15b from the first intermediate image forming point 13b exits the exit reflecting surface of the parallel prism 18b to be formed at a second intermediate image forming point 21b. The image is guided to an eyepiece optical system 22b incorporated in the ocular housing 5b. The optical path constitutes the observation optical axis OR of the surgical microscope.

The interpupillary adjustment housing 4b is supported by the movable housing 16 such that it can be rotated about an axis that substantially agrees with the axis of light exiting the triangular prism 12b. The interpupillary adjustment housing 4b is prevented from moving in the axial direction by a stopper member. This structure along with the parallel prism 18b forms a so-called Siedentopf-type interpupillary adjustment structure.

FIG. 3 shows the lens body 4 of the surgical microscope. The part indicated by a broken line above the lens body 4 is the binocular eyepiece tube section described with reference to FIGS. 1 and 2. The lens body 4 incorporates the objective optical system 24, the variable power optical system 25, and the eyepiece optical systems (the binocular eyepiece tube section), and there is provided a pair of left and right optical paths. The objective optical system 24 is provided with a focus change mechanism (not shown) and a focal length detection sensor (not shown). Further, the variable power optical system 25 is provided with a power change mechanism (not shown) and a variable power detection sensor (not shown). The lens body 4 is provided with a superimposing section having an optical path inserting portion 26 constituted by a half mirror and an image inserting optical system 27. The image inserting optical system 27 converts a beam of light emitted from an image superposing monitor 69a into an afocal beam of light which then enters the optical path inserting portion 26. Reference numeral 128a represents a cable for transmitting a video signal to the image superposing monitor 69a. The superposing section is used to superpose separate images on an image obtained by the microscope.

FIG. 4 is a perspective view of an LCD optical system provided in the eyepiece tubes. The LCD optical system is used to allot a part of the field of view of the microscope for display of separate images. Small LCD monitors 24a and 24b display images obtained by the endoscope or preoperative images as electronic images under control of a controller (not shown). Image forming lenses 25a and 25b that are projection optical systems are provided on exit optical axes of the LCD monitors 24a and 24b and fixed such that they form images from the LCD monitors 24a and 24b on top surfaces of the prisms 14a and 14b. The LCD monitor 24a, the image forming lens 25a, and the prism 14a constitute an LCD optical system 26a. Similarly, the LCD monitor 24b, the image forming lens 25b, and the prism 14b constitute an LCD optical system 26b.

The LCD optical systems 26a and 26b are fixed to a fixing plate 27.

The fixing plate 27 has holes 27a and 27b through which beams of light from the objective optical system 24 of the microscope pass. The fixing plate 27 is fixed on an X table 29a of an X-Y table 28a that is a driving section, and the X table 29a is provided such that it can move on a plane orthogonal to an optical axis in X and Y directions. When the X-Y table 28a moves, the prisms 14a and 14b move relative to a beam of light from the surgical microscope. This results in a movement of the position of the in-field image 145 shown in FIG. 8.

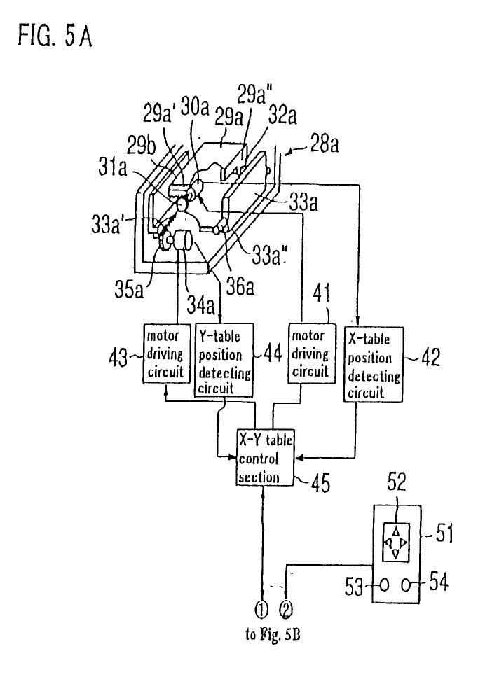

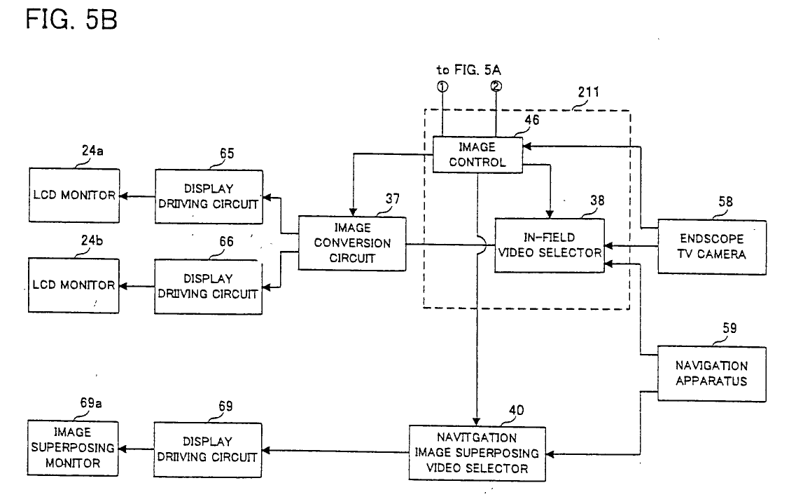

FIG. 5A is a perspective view showing an internal structure of the X-Y table 28a and a block diagram of a control system. FIG. 5B is a block diagram of a control system. Most of the circuits in the control system are provided in the lens body 104. The X table 29a has a rack section 29a′ and a bearing section 29a″. The rack section 29a′ is engaged with a pinion gear 31a secured to a rotating shaft of a motor 30a. A guide shaft 32a extends through the bearing section 29a″. The motor 30a and the guide shaft 32a are secured to a Y table 33a.

The Y table 33a has a rack section 33a′ and a bearing section 33″. The rack section 33a′ is engaged with a pinion gear 35a secured to a rotating shaft of a motor 34a. A guide shaft 36a extends through the bearing section 33a″.

Each of the motors 30a, 34a incorporate an encoder, and they are electrically connected to the control system which will be described later. Specifically, the motor 30a is connected to a motor driving circuit 41, and its encoder is connected to an X table position detecting circuit 42. The motor 34a is connected to a motor driving circuit 43, and its encoder is connected to a Y table position detecting circuit 44. The motor driving circuit 41, the X table position detecting circuit 42, the motor driving circuit 43, and the Y table position detecting circuit 44 are connected to an X-Y table control section 45.

An operating section (operation input section) 51 operated by an operator is provided with a four direction X-Y switch 52 for operating the X-Y table 28a, observing section select switch 53, and a display select switch 54.

The operating section 51 is connected to an image control section 46. The image control section 46 is connected to the X-Y table control section 45, an image conversion circuit section 37, and an in-field video selector 38. The in-field video selector 38 is connected to an endoscope TV camera 58 (which is contained in a TV camera containing section 125 in FIG. 6) and a navigation apparatus 59 (see FIG. 6). The image conversion circuit section 37 is connected to the LCD monitor 24a through a display driving circuit 65 and connected to the LCD monitor 24b through a display driving circuit 66. The image control section 46 and the in-field video selector 38 constitute a display control section 211.

A navigation image superposing video selector 40 is also connected to the image control section 46. The navigation image superposing video selector 40 is connected to the navigation apparatus 59.

The navigation image superposing video selector 40 is also connected to the image superposing monitor 69a provided in the lens body 4 through the display driving circuit 69.

FIG. 6 is a perspective view showing a general system configuration of the surgical microscopic apparatus. As shown in FIG. 6, the surgical microscopic apparatus has a surgical microscope 101 that is a stereoscopic microscope, an endoscope 121 that is a rigid endoscope for obtaining an observation image different from an image observed with the surgical microscope 101, and a display monitor 141 as a display section for displaying images observed with the surgical microscope 101 and the endoscope 121.

The surgical microscope 101 is provided with a stand 102, and a balance arm 103 disposed on top of the stand 102, and a lens body 104 supported by the balance arm 103.

A plurality of movable arms are coupled to the balance arm 103 with six rotating shafts 105a to 105f. Further, each of the rotating shafts 105a to 105f is provided with an electromagnetic lock (not shown) for switching a locked state in which the rotating position of the rotating arm is fixed and an unlocked state in which such locking is canceled. The position of the lens body 104 supported by the balance arm 103 can be spatially moved freely through rotations of the rotating arms about the respective rotating shafts 105a to 105f. It can be freely moved and stopped through an operation of switching the locking and unlocking of the electronic locks.

The lens body 104 is provided with a sensor arm 106 and a grip 107 for an operation of positioning the lens body 104. The grip 107 is provided with operating switches for focus adjustment, power change operations, and arm operations.

The surgical microscope 101 incorporates a lens body control section 111 and an arm control section 112. Each of the switches on the grip 107 is connected to the lens body control section 111 and the arm control section 112. Further, a foot switch 113 having switches for focus adjustment and power changing similar to the switches on the grip 107 is connected to the lens body control section 111 and the arm control section 112.

The endoscope 121 is supported by a scope holder 122 that is mounted on an operating table (not shown). The scope holder 122 is constituted by a multiarticular arm having a plurality of movable arms 123, and joints between the movable arms 123 are rotatably linked. The endoscope 121 is movably supported by the scope holder 122.

Further, each of the rotating sections of the scope holder 122 is provided with an electromagnetic lock for switching a locked state in which the rotating position of the movable arm 123 of the scope holder 122 is fixed and an unlocked state in which the locking of the rotating position is canceled. The endoscope 121 is supported such that it can be moved in accordance with the operation of switching the locking and unlocking of the electromagnetic locks of the scope holder 122.

The electromagnetic lock at each of the rotating section is connected to a scope holder driving/controlling section 124. A switch 122A for operating the electromagnetic locks is provided at an end of the scope holder 122. The switch 122A is connected to the scope holder driving/controlling section 124. Further, the TV camera containing section 125 for containing the TV camera 58 and a substantially V-shaped endoscope sensor arm 126 are mounted to the endoscope 121.

There is also provided a digitizer 134 as a photographic apparatus for detecting observing positions of the surgical microscope 101 and the endoscope 121. The digitizer 134 detects the observing positions of the surgical microscope 101 and the endoscope 121 by detecting the sensor arm 106 of the lens body 104 of the surgical microscope 101 and the sensor arm 126 of the endoscope 121.

The navigation apparatus 59 is connected to the digitizer 134. The navigation apparatus 59 incorporates a memory device for diagnostic images (including data for generating preoperative images) and also has a processing section for correlation with diagnostic images. The display monitor 141 and an interface unit 136 are also connected to the navigation apparatus 59. Image information from the digitizer 134 is input to the navigation apparatus 59, and correlation between the image information and reference indices attached to the head of the patient is calculated by the navigation apparatus 59.

Using the above-described configuration, an operator operates the balance arm 103 to fix the lens body 104 of the surgical microscope in a desired position. Further, the operator rotates the movable housing 16 (FIG. 1) of the lens body 104 about the axis O to position the eyepiece optical systems 22a and 22b at the eyes of the operator. At this time, the image rotator prisms 10a and 10b in the fixed housing 7 rotate halfway the rotation of the movable housing 16 about the axis O.

Optical processes in the surgical microscope will now be described. Light from an operated part enters the image forming lenses 8a and 8b in FIG. 1 through the optical system in the lens body 4 in FIG. 3. Left and right beams of light that have exited the image forming lenses 8a and 8b pass through the image rotator prisms 10a and 10b, which corrects a rotation of the image attributable to the rotation of the movable housing 16 about the axis O. Thereafter, the beams of light are reflected by the prisms 11a and 11b and the triangular prisms 12a and 12b and are formed into images at the first intermediate image forming points 13a and 13b.

Then, the beams of light are relayed by the relay lenses 15a and 15b, reflected by the parallel prisms 18a and 18b, and thereafter formed into images again at the second intermediate image forming points 21a and 21b. The beams of light are then guided to the eyepiece optical systems 22a and 22b to be used by the operator for stereoscopic observation at a desired magnification. When stereoscopic observation is disabled by a deviation between the distance between the left and right observation optical axes OL and OR and the interpupillary distance of the operator, the eyepiece tubes 4a and 4b are rotated to perform interpupillary adjustment for adjusting the left and right observation optical axes OL and OR to the interpupillary distance of the operator.

When it is desired to observe an image observed by the endoscope, a preoperative image obtained by CT or MR, or a preoperative image created from preoperative data obtained by CT or MR (a navigation image that is a preoperative image) simultaneously with an image from the surgical microscope using the in-field display section, the operator operates the operating section 51 (FIG. 5A) to display any of the images (e.g., an image obtained by the endoscope) on the LCD monitors 24a and 24b (FIG. 4). Lights emitted by the LCD monitors 24a and 24b are formed into images on the top surfaces of the prisms 14a and 14b by the image forming lenses 25a and 25b. Since the top surfaces of the prisms 14a and 14b are located in the vicinity of the first image forming points 13a and 13b, the images obtained by the endoscope are displayed in the fields of observation of the surgical microscope.

The display position of an in-field image of the surgical microscope can preferably be moved. For example, when the observing section select switch 53 of the operating section 51 is turned on with the endoscope TV camera 58 (endoscope image) selected in FIGS. 5A and 5B, the four directional switch 52 enters a step mode, and the switch enters a free mode when the select switch is turned off. The four directional switch 52 is turned on to drive the X-Y table 28a. When the motor 30a is driven through the motor driving circuit 41 by operating the four directional switch 52, the pinion gear 31a rotates. The bearing section 29a″ of the X table 29a is supported by the guide shaft 32a secured to the Y table 33a. Therefore, the X table 29a moves in the X-direction along the guide shaft 32a when a rotary force of the pinion gear 31a is received by the rack section 29a′. This also causes the fixed plate 27a (FIG. 4) fixed on the X table 29a to move, and the prism 14a (FIG. 4) moves on the first intermediate image forming point 13a to move the endoscope image in the X-direction consequently.

When the motor 34a is driven through the motor driving circuit 43 by operating the four directional switch 52, the pinion gear 35a rotates. The bearing section 33a″ of the Y table 33a is supported by the guide shaft 36a secured to the X table 29a. Therefore, the Y table 33a moves in the Y-direction along the guide shaft 36a when a rotary force of the pinion gear 35a is received by the rack section 33a′. This also causes the fixed plate 27a fixed on the Y table 33a to move, and the prism 14a moves on the first intermediate image forming point 13a to move the endoscope image in the Y-direction consequently.

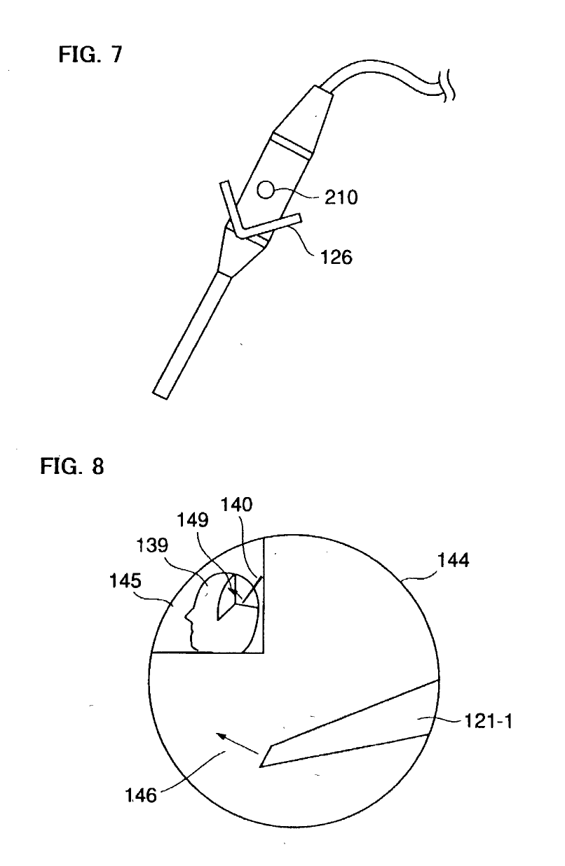

FIG. 7 shows the endoscope 121 described with reference to FIG. 6 which is provided with the endoscope sensor arm 126 and a switch 210. The switch 210 transmits signals for switching images from the LCD monitor 24a and the LCD monitor 24b that display images in the in-field display section to the image control section 46. The switch 210 is connected to the endoscope TV camera 58 in FIG. 5B, and the endoscope TV camera 58 is connected to the image control section 46.

An operator may conduct observation with the endoscope 121 held in his or her hand instead of supporting the endoscope 121 with the scope holder 122 as shown in FIG. 6.

Operations of the present surgical microscopic apparatus will now be described.

An operator starts observing an operated part by moving the lens body 104 of the surgical microscope 101 to a desired position. When a part to be observed under operation is difficult to observe with the surgical microscope 101 or located at a dead angle to the observing range of the surgical microscope 101, the endoscope 121 is moved to the operated part. When the endoscope 121 is used, an assistant inputs a viewing angle of the endoscope 121 at a workstation installed in the navigation apparatus 59 in advance.

When the operator presses the switch provided on the operating section 51 for enabling display in the in-field display section or the switch 210 provided on the endoscope 121 to observe an image observed by the endoscope 121 during observation using the surgical microscope 101, the image control section 46 detects that the switch has been pressed.

An order for switching of images displayed in the in-field display section is recorded in the image control section 46. The images are switched by pressing the switch 210 provided on the endoscope 121. As thus described, the present surgical microscopic apparatus is set such that video signals connected to the display control section 211 are sequentially displayed in the in-field display section in the order of switching recorded in the image control section 46 through the operation on the switch 210.

In the present embodiment, video signals of a preoperative image having a range wider than the field of view of the endoscope (e.g., a navigation image for the entire head of a patient) and a preoperative image having the same range as the range of observation of the endoscope are supplied from the navigation apparatus 59 through different signal lines respectively. Further, a video signal of an endoscope image is also supplied from the TV camera 58 in the endoscope. The three video signals are sequentially selected one by one in the order recorded in the image control section 46 through operations on the switch 210.

The image control section 46 transmits signals that designates a video signal to be selected to the in-field video selector 38.

A method of selecting a video signal will now be described. The two video signals form the navigation apparatus 59 and the video signal from the endoscope TV camera 58 are transmitted to the in-field video selector 38. Each of cables for transmitting the signals is connected to a connector portion (not shown) of the display control section 211. Specifically, the navigation image (of the entire head) having a wide range transmitted from the navigation apparatus 59, the image observed by the stereoscopic endoscope having the same range as the range of observation of the endoscope transmitted from the navigation apparatus 59 (the two video signals from the navigation apparatus), and the image observed by the endoscope (the video signal from the endoscope TV camera 58) are connected to separate connectors respectively in the above-described order (they are sequentially connected to the connector portion of the display control section 211 starting with the connector at one end thereof). The in-field video selector 38 determines the connector from which the video signal is selected according to an instruction from the image control section 46. The in-field video selector 38 selects a connector in practice.

The digitizer 134 detects the observing position of the endoscope 121 from the position of the endoscope sensor arm 126 and detects the observing position of the lens body 104 of the surgical microscope from the position of the surgical microscope sensor arm 106.

The position information is transmitted to the navigation apparatus 59. The navigation apparatus 59 calculates the observing position and viewing direction of the endoscope for the preoperative image and the viewing direction of the endoscope in the image observed by the surgical microscope using the workstation provided therein.

As a result of those operations, as shown in FIG. 8, in the in-field display section of the surgical microscope, an image 145 is displayed which is a preoperative image 139 superposed with an observing position 140 of the endoscope 121 and a viewing direction 149 of the endoscope 121 for the preoperative image 139 (the image 145 is displayed on the monitors 24a and 24b in FIG. 4 and is introduced into the field of view of the surgical microscope by the optical system in FIG. 4). An arrow 146 indicating the viewing direction of the endoscope 121 is superposed and displayed on an image 144 observed by the surgical microscope by the superposing section (the arrow 146 is displayed on the monitor 69a in FIG. 3 and superposed on the field of view of the surgical microscope by the image inserting optical system 27 and the half mirror 26). Reference numeral 121-1 represents an image of the endoscope 121.

The operator checks the observing position of the endoscope 121 from the image 145. The operator also compares the image 145 and the arrow 146 to check where the observing position of the endoscope in the field of view of the surgical microscope is located in the preoperative image. Thus, the position of the endoscope 121 in the head as a whole can be understood from the preoperative image 139 having a range wider than the field of view of the surgical microscope.

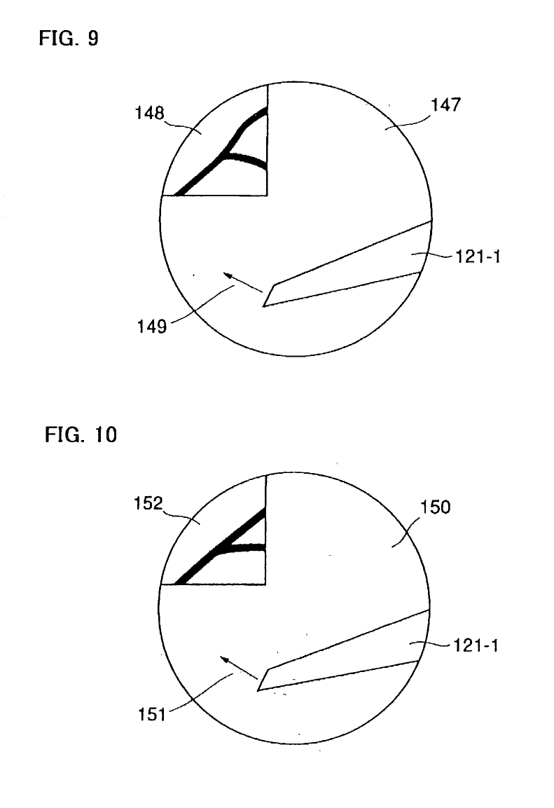

A next press on the switch 210 causes the image control section 46 to transmit an instruction to the in-field video selector 38 to display a stereoscopic preoperative image having the same range as the observing range of the endoscope as the image to be displayed in the in-field display section. As a result, a stereoscopic preoperative image 148 having the same range as the observing range of the endoscope is displayed in the in-field display section as shown in FIG. 9. An image on which an arrow indicating the viewing direction 149 of the endoscope 121 is superposed is displayed as an image 147 observed by the surgical microscope. From this image displayed in the field of view, the observing position of the endoscope can be checked in detail in a range smaller than that shown in FIG. 8.

When it is confirmed that the current position of the endoscope 121 is a desired observing position, the operator then presses the switch 210 to switch the image displayed in the in-field display section in order to start observation with the endoscope. Specifically, when the operator presses the switch 210, the image control section 46 detects the press, and a signal is transmitted from the image control section 46 to cause the in-field video selector 38 to switch the image, whereby the image displayed in the field of view is switched to an image 152 observed by the endoscope (FIG. 10). In FIG. 10, reference numeral 151 represents an arrow indicating the viewing angle of the endoscope 121, and the reference numeral 150 represents an image observed by the microscope just as in FIGS. 8 and 9.

Further presses on the switch 210 of the endoscope cause the image displayed in the in-field display section to be switched from FIG. 10 to FIG. 9 and then to FIG. 8 (the image is thus switched in an order starting with FIG. 8, followed by FIG. 9, FIG. 10, FIG. 9, FIG. 8, FIG. 9, FIG. 10, and so on). Each time the switch 210 is pressed, the image displayed in the field of view changes in the above order.

While the image is switched with the switch 210 provided on the endoscope 121 in the above description, the image may alternatively be switched using a switch provided on the foot switch 113 of the surgical microscope 101 or a switch provided on the operating section 51.

The first embodiment makes it possible to start observation with the endoscope after checking a preoperative image having a wide range and then checking the observing position of the endoscope in a smaller range according to the order of displaying recorded images in the field of view of an image observed by the microscope. This makes it possible to confirm the position of the endoscope easily from a preoperative image when the endoscope is inserted.

Since the viewing direction of the endoscope 121 superposed on a microscope image and the observing direction of the endoscope 121 in a preoperative image can be simultaneously recognized, the orientation of the endoscope 121 is facilitated.

Further, it is required to provide only one switch on the endoscope 121, and the display control section 211 has no feature for device identification, which simplifies the configuration of an apparatus and therefore reduces the cost of the same.

In a second embodiment of the invention, there are two switches for switching the image displayed in the in-field display section, and the image is switched by the switches in respective orders that are opposite to each other.

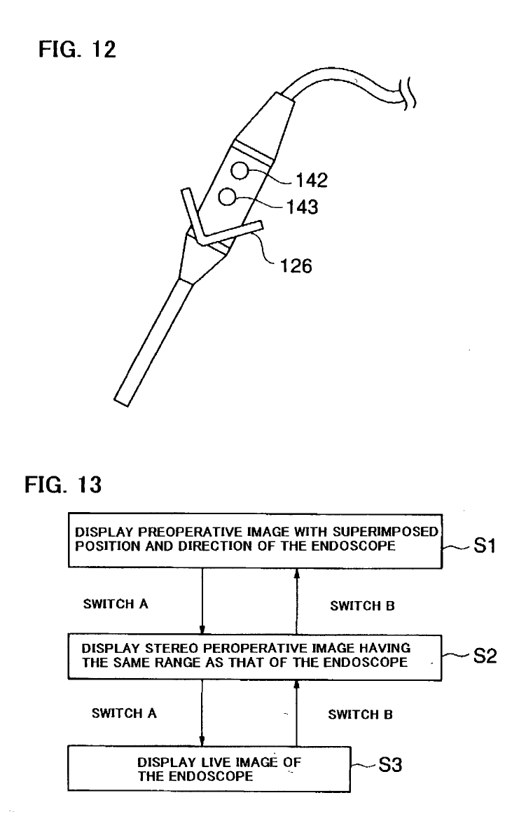

The second embodiment will be described with reference to FIGS. 1 to 10 and FIG. 12, the description being focused only on differences from the first embodiments. In the second embodiment, a switch A 142 and a switch B 143 are provided on an endoscope 121 as shown in FIG. 12. An image switching order for the switch A 142 and an image switching order for the switch B 143 are recorded in an image control section 46 of a display control section 211 as shown in FIG. 5B. The image switching order for the switch A 142 and the image switching order for the switch B 143 are set in reverse.

Operations of the second embodiment will now be described.

An image is displayed in the in-field display section in the same way as that in the first embodiment. In an in-field display section, there is displayed an image 145 that is a preoperative image 139 on which an observing position 140 of an endoscope 121 for the preoperative image 139 and a viewing direction 149 of the endoscope 121 are superposed, as shown in FIG. 8.

When the switch A 142 is pressed, a stereoscopic preoperative image 148 having the same range as the observing range of the endoscope is displayed in the in-field display section (FIG. 9). Another press on the switch A 142 caused an image 152 observed by the endoscope to be displayed in the in-field display section (FIG. 10).

Thus, the image displayed in the field of view is switched in an order starting with FIG. 8, followed by FIG. 9, FIG. 10, FIG. 8, FIG. 9, and then FIG. 10 by pressing the switch A142.

In the images shown in FIGS. 8 to 10, an arrow indicating the viewing direction of the endoscope 121 is superposed on an image observed by a surgical microscope 101 by a superposing section.

To check an image of the entire head of a patient from a preoperative image when a stereoscopic operative image 148 having the same range as the observing range of the endoscope is displayed in the in-field display section as shown in FIG. 9, the switch B 143 is pressed to switch the image. Then, the operator can observe the preoperative image 139 shown in FIG. 8.

When the switch B 143 is pressed with an image observed by the endoscope as shown in FIG. 10 displayed in the in-field display section, the display in the in-field display section is switched to an observed image in which the stereoscopic preoperative image 148 having the same range as the observing range of the endoscope is displayed as shown in FIG. 9.

Thus, the image displayed in the field of view is switched in an order starting with FIG. 9, followed by FIG. 8, FIG. 10, FIG. 9, and then FIG. 8 by pressing the switch B 143.

In addition to the advantages of the first embodiment, the second embodiment makes it possible to check the previous image easily because switches for switching images in opposite directions are provided, and this leads to a reduction of operating time.

In a third embodiment of the invention, again, two switches are provided on an endoscope to switch images displayed in an in-field display section. The third embodiment will be described with reference to FIGS. 1 to 4 and FIGS. 6 to 13.

The description will be focused on only differences from the first embodiment.

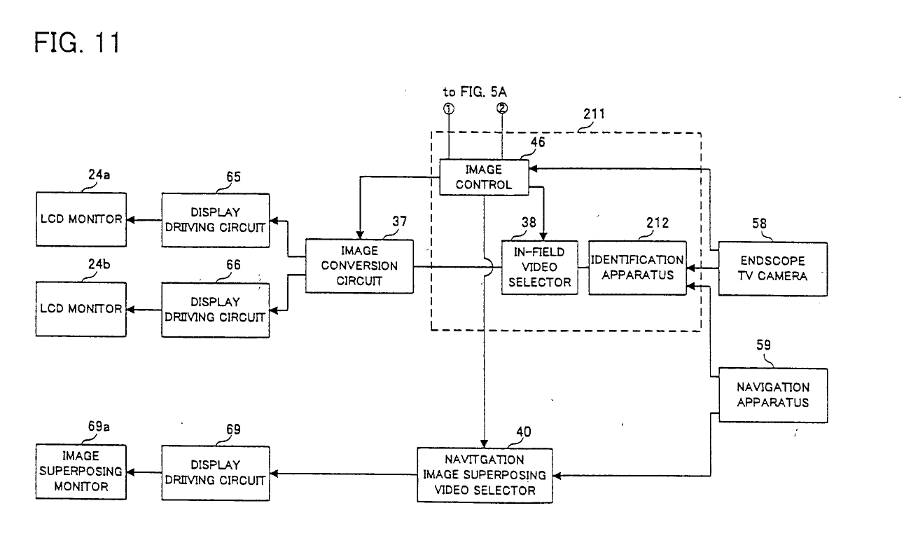

An endoscope 121 in FIG. 12 is provided with an endoscope sensor arm 126, a switch A 142, and a switch B 143. The switch A 142 and the switch B 143 transmit signals for switching images on an LCD monitor 24a and an LCD monitor 24b for displaying images in the in-field display section to an image control section 46. The switch A 142 and the switch B 143 are connected to an endoscope TV camera 58 in FIG. 11, and the endoscope TV camera 58 is connected to a display control section 211. An identification apparatus 212 for identifying types of images is also connected to the display control section 211.

An operator may conduct observation with the endoscope 121 held in his or her hand instead of supporting the endoscope 121 with a scope holder 122 as shown in FIG. 6.

As shown in FIG. 11, the image control section 46 in which orders for displaying images are recorded, an in-field video selector 38, and the identification apparatus 212 as an identification section are provided in the display control section 211.

Operations of the third embodiment will now be described.

An operator starts observing an operated part by moving a lens body 104 of a surgical microscope 101 to a desired position. When a part to be observed under operation is difficult to observe with the surgical microscope 101 or located at a dead angle to the observing range of the surgical microscope 101, the operator moves the endoscope 121 to the operated part. When the endoscope 121 is used, an assistant inputs a viewing angle of the endoscope 121 at a workstation installed in a navigation apparatus 59 in advance.

When the operator presses a switch provided on an operating section 51 for enabling display in the in-field display section or the switch A 142 or switch B 143 provided on the endoscope 121 to observe an image observed by the endoscope 121 during observation using the surgical microscope 101, the image control section 46 detects that the switch has been pressed.

Orders for switching of images displayed in the in-field display section are recorded in the image control section 46. The displayed images are switched by pressing the switch A 142 and B 143 provided on the endoscope.

FIG. 13 is a flow chart for explaining an order of switching of images displayed in the in-field display section that occurs when the switch A 142 or the switch B 143 is pressed. First, an image is displayed in which the position and viewing direction of the endoscope 121 are superposed on a preoperative image having a wide range (step S1). Then, a press on the switch A 142 causes a stereoscopic preoperative image having the same range as the observing range of the endoscope 121 to be displayed (step S2). Another press on the switch A 142 caused an actual endoscope image to be displayed (step S3). When the switch B 143 is then pressed, the process returns to step S2 at which the stereoscopic preoperative image having the same range as the observing range of the endoscope 121 is displayed. Another press on the switch B 143 causes display of the image in which the position and viewing angle of the endoscope 121 are superposed on the preoperative image having a wide range (step S1).

The image control section 46 transmits a signal to the in-field video selector 38 to display a preoperative image recorded in a navigation apparatus 59 in the in-field display section,

The endoscope TV camera 58 and the navigation apparatus 59 generate identification signals by identification signal generating sections (not shown) and transmit respective video signals and the identification signals identifying them as the endoscope TV camera 58 or navigation apparatus 59 to the identification apparatus 212. The identification apparatus 212 identifies the video signals from the endoscope TV camera 58 and the navigation apparatus 59 from the identification signals.

A digitizer 134 detects the observing position of the endoscope 121 from the position of the endoscope sensor arm 126 and detects the observing position of the surgical microscope 101 from the position of a surgical microscope sensor arm 106. The position information is transmitted to the navigation apparatus 59. A workstation provided in the navigation apparatus 59 calculates the observing position and viewing direction of the endoscope for the preoperative image and the viewing direction of the endoscope in the image observed by the surgical microscope.

The identification apparatus 121 identifies the signal from the navigation apparatus 59 and transmits the video signal to the in-field video selector 38. In the in-field display section, an image 145 is first displayed which is a preoperative image 139 superposed with an observing position 140 of the endoscope 121 and a viewing direction 149 of the endoscope 121 (for the preoperative image 139). An arrow 146 indicating the viewing direction of the endoscope 121 is superposed and displayed on an image 144 observed by the surgical microscope by the superposing section (FIG. 8).

The operator checks the observing position of the endoscope 121 from the image 145. The operator also compares the image 145 and the arrow 146 to check where the observing position of the endoscope in the field of view of the surgical microscope is located in the preoperative image. Thus, the position of the endoscope in the head as a whole can be understood from the preoperative image having a wide range.

The operator then presses the switch A 142 to switch the image to be displayed in the in-field display section in order to start observation with the endoscope 121. When the operator presses the switch A 142, the image control section 46 detects the press, and a signal instructing image switching is transmitted from the image control section 46 to the identification apparatus 212.

The identification apparatus 212 transmits a signal to the navigation apparatus 59 to display a stereoscopic preoperative image having the same range as the field of view of the endoscope generated by the navigation apparatus 59. The navigation apparatus 59 processes the viewing direction of the endoscope 121 input in advance, the observing position of the endoscope 121 detected by the digitizer 134, and the observing position of the surgical microscope 101 with the workstation provided in the navigation apparatus 59 to generate a stereoscopic preoperative image having the same range as the field of view of the endoscope. It transmits the stereoscopic preoperative image to the in-field video selector 38 to display a stereoscopic preoperative image 148 having the same range as the field of observation of the endoscope in the in-field display section (FIG. 9).

The operator can observe the image 148 to closely check the observing position of the endoscope 121 in a range smaller than that of the image 145 shown in FIG. 8 and can therefore confirm that the position is a desired observing position. Further, the operator observes an image 152 actually observed by the endoscope 121 by pressing the switch A 142 further (FIG. 10).

By pressing the switch B 143 according to the procedure described with reference to FIG. 13, the image switching direction is reversed from that in the case of the switch A 142.

While the image switching is carried out here using the switch A 142 and the switch B 143 provided on the endoscope 121, the image may be switched using a switch provided on a foot switch 113 of the surgical microscope 101 or a switch provided on an operating section 151.

While the identification apparatus 212 transmits a signal to cause the navigation apparatus 59 to switch the image displayed as in FIG. 9, three images (a preoperative image having the same range as the range of view of the endoscope provided by the navigation apparatus 59, an image that is a preoperative image having a wide range provided by the navigation apparatus 59 on which the observing position and viewing direction of the endoscope 121 are superposed, and an image observed by the endoscope) may be input to the display control section 211, and those images may be identified by the identification apparatus 212 and transmitted to the in-field video selector 38.

According to the third embodiment, there is no concern about the position to insert a connector because images are identified from identification signals. A preoperative image having a wide range is switched to a preoperative image having a smaller range according to a recorded order of displaying images in the field of view of an image observed by the, surgical microscope. It is therefore possible to check the position of the endoscope 121 from the preoperative images when it is inserted with the position of the same in the entire head of a patient recognized. Since the images can be switched with the switches A 142 and B 143 provided on the endoscope 121, the operator can obtain a desired image.

Further, since the viewing direction of the endoscope 121 superposed on a microscope image and the observing direction of the endoscope 121 in a preoperative image can be simultaneously recognized, the orientation of the endoscope 121 is facilitated.

A fourth embodiment of the invention is characterized in that control over images displayed in an in-field display section is carried out in different ways. The fourth embodiment will be described with reference to FIG. 1, FIGS. 8 to 10, and FIG. 13. The description will be focused on only differences from the third embodiment.

A connecting section A and a connecting section B that are not shown are provided in an in-field video selector 38, and the connecting sections A and B have connectors in different configurations. An endoscope TV camera 58 is connected to the in-field video selector 38 at the connecting section A, and a navigation apparatus 59 is connected to the same at the connecting section B.

Operations of the fourth embodiment will now be described.

The description will be made only on display image control for displaying images in the in-field display section. The flow of image switching is the same as that in FIG. 13.

The endoscope TV camera 58 is connected to the connecting section A (not shown) of the in-field video selector 38, and the navigation apparatus 59 is connected to the connecting section B (not shown) of the in-field video selector 38. The connecting sections A and B are constituted by different types of connectors. For example, the connecting section A is a Y-C connector, and the connecting section B is an RGB connector.

The order of switching images recorded in an image control section 46 depends on the connector types. When the switch A 142 is pressed, an image from the connecting section B (navigation apparatus) is displayed in the in-field display section. First, the image shown in FIG. 8 is displayed.

Another press on the switch A 142 causes the in-field video selector 38 to transmit a signal to the navigation apparatus 59, and the navigation apparatus 59 switches the signal to be sent to the in-field video selector 38. The image switched by the navigation apparatus 59 is displayed in the in-field display section (FIG. 9).

When the switch A 142 is pressed next, the image control section 46 transmits a signal to the in-field video selector 38 to cause it to display an image from the connecting section A. An image similar to FIG. 10 is displayed.

In addition to the advantages of the third embodiment, the fourth embodiment eliminates the need for the configuration for transmitting identification signals from the endoscope TV connector 58 and the navigation apparatus 59 to the in-field video selector 38. Since this eliminates the configuration to allow the in-field video selector 38 to judge identification signals, a simple configuration can be employed. This makes it possible to provide a compact apparatus that can be easily transported. The apparatus can be also provided at a low cost.

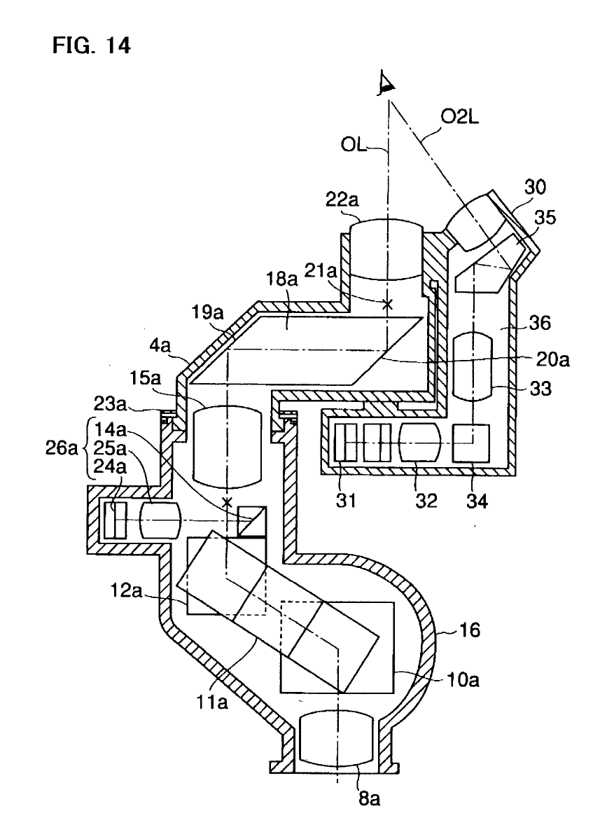

A fifth embodiment of the invention is characterized in that an image in an in-field display section and an image from an observation optical system 36 are exchanged through a operation of a switch provided on an endoscope. The fifth embodiment will be described with reference to FIGS. 14 to 20.

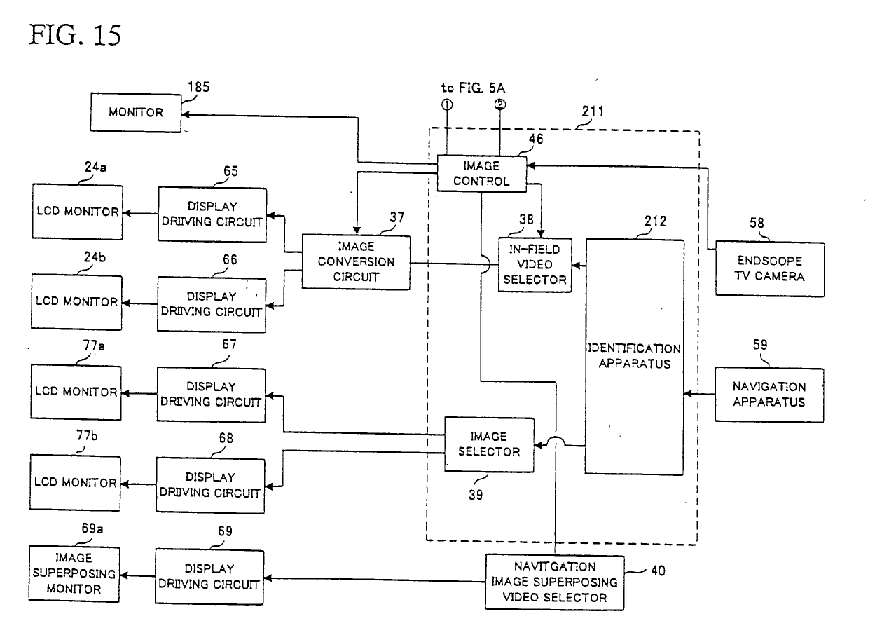

The fifth embodiment is a combination of the configuration of the third embodiment and configurations in FIGS. 14 and 15.

Referring to FIG. 14, a second eyepiece housing 30 for containing a second observation optical system 36 is added to the configuration in FIG. 2. The second observation optical system 36 has small LCD monitor boards 31, relay optical systems 32 and 33, prisms 34 and 35, and a second eyepiece optical system.

In FIG. 15, several elements are added to the configuration in FIG. 11. The elements added are LCD monitors 77a and 77b for the second observation optical system, display driving circuits 67 and 68, and an image selector 39 and a monitor 185 for observing an image from the second observation optical system. The LCD monitors 77a and 77b are incorporated in the left and right LCD monitor boards 31 in FIG. 14.

Operations of the fifth embodiment will now be described.

The description will be made only on differences from the third embodiment. When an endoscope image and a preoperative image are to be displayed in the field of view of a surgical microscope 101, a switch A 142 provided on an endoscope 121 is operated. At this time, since a signal is also transmitted from an image control section 46 to the image selector 39 for image observation to cause the LCD monitors 77a and 77b to start operating, an image can be also observed with the second observation optical system. Control over the image selector 39 for image observation is similar to control over the in-field video selector 38 carried out by the image control section 46 in the first embodiment.

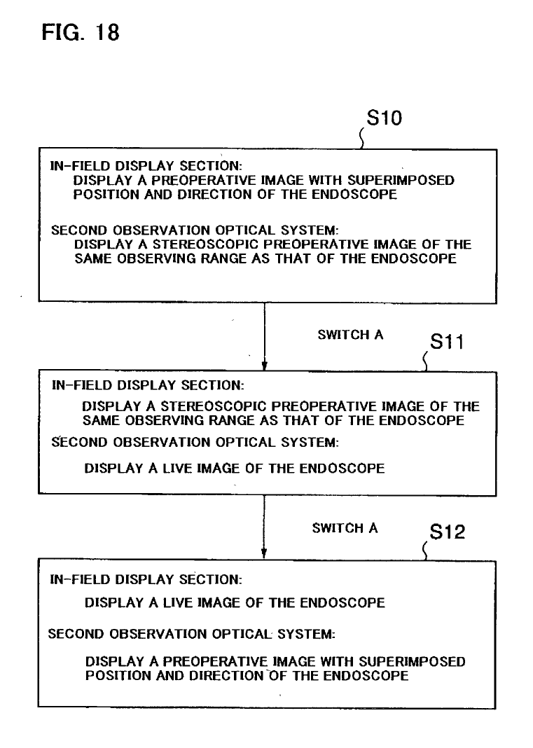

FIG. 18 shows a flow of image display caused by the operation of the switch A 142 and switch B 143.

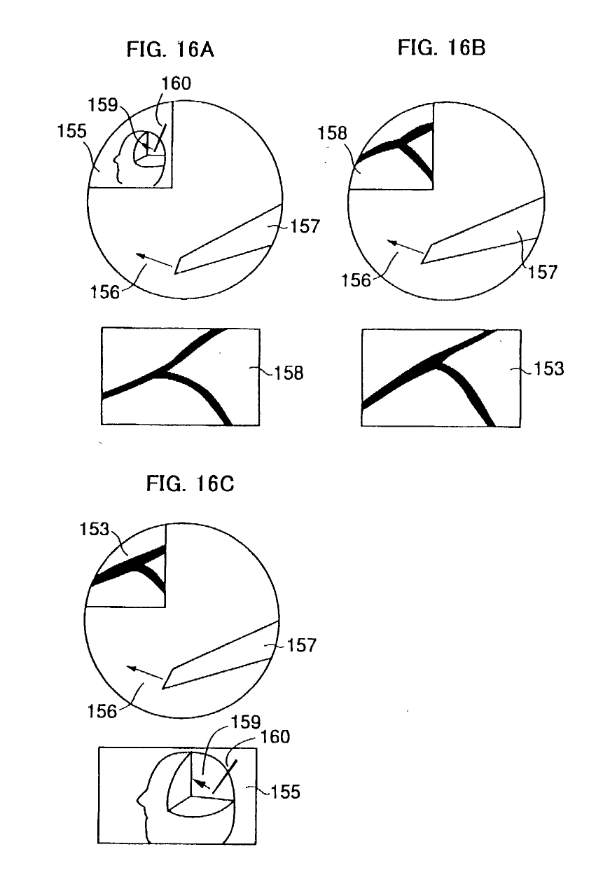

At the beginning of the observation, as shown in FIG. 16A, the in-field display section displays an image 155 that is a preoperative image having an observing position 160 and a viewing direction 159 of the endoscope 121 superposed thereon; the second observation optical system 36 displays a stereoscopic preoperative image 158 having the same range as the observing range of the endoscope; and an image 157 of the endoscope 121 and a viewing direction 156 of the endoscope 121 are superposed on an image observed by the microscope (step S10).

An operator checks the observing position of the endoscope 121 relative to the head of a patient as a whole from the image 155 in the in-field display section that is a preoperative image having the observing position 160 and the viewing direction 159 of the endoscope 121 superposed thereon. Further, the operator checks the observing position of the endoscope in more detail from the stereoscopic preoperative image having the same range as the observing range of the endoscope displayed by the second observation optical system 36. When it is confirmed that the endoscope 121 is in a proper position, the operator fixes the position of the endoscope 121.

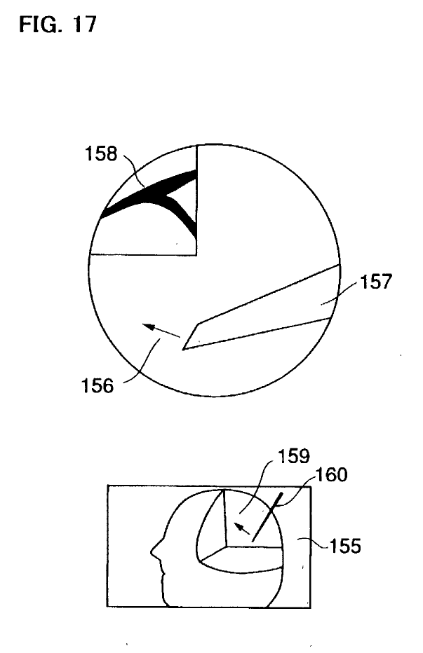

The operator presses the switch B 143 when it is desired to look at the image 155 in the in-field display section that is a preoperative image having a wide range superposed with the observing position 160 and the viewing direction 159 of the endoscope 121 on a greater screen. This causes the image 155 displayed in the in-field display section and the image 158 displayed by the second observation optical system 36 change places with each other (FIG. 17). This allows the operator to check the observing position of the endoscope by looking at the image 155 having the observing position 160 and viewing direction 159 of the endoscope superposed thereon on a greater screen.

When the operator presses the switch A 142 next, the stereoscopic preoperative image 158 having the same range as the observing range of the endoscope in the in-field display unit, and an actual endoscope image 153 is displayed by the second observing optical system 36 (FIG. 16B, and step S11 in FIG. 18). The operator checks the part observed by the endoscope by comparing the stereoscopic preoperative image 158 having the same range as the observing range of the endoscope displayed in the in-field display unit and the actual image 153 observed by the endoscope displayed by the second observing optical system 36.

When the operator confirms that the endoscope has been moved to a desired position, the operator presses the switch A 142 further. Then, the endoscope image 153 is displayed in the in-field display unit, and the second observing optical system 36 displays the image 155 that is a preoperative image having the observing position 160 and the viewing direction 159 of the endoscope superposed thereon (FIG. 16c, step S12 in FIG. 18). The operator treats the operated part while observing those images.

Thus, an image in the in-field display section and an image from the second observing optical system can be replaced with each other in respective modes of image display (FIGS. 16A, 16B, and 16C) by pressing the switch B 143.

While the images are switched with the switches A 142 and B 143 provided on the endoscope 121 in the above example, the images may be switched with a switch provided at a foot switch 113 of the surgical microscope 101 or a switch provided on an operating section 51.

In the fifth embodiment of the invention, the setting of the order of display screens can be changed. The setting of the order of display screens is changed using a setting mode button (not shown) provided on the operating section 51 as a setting change section and a switch 52. First, the setting mode button of the operating section 51 is pressed.

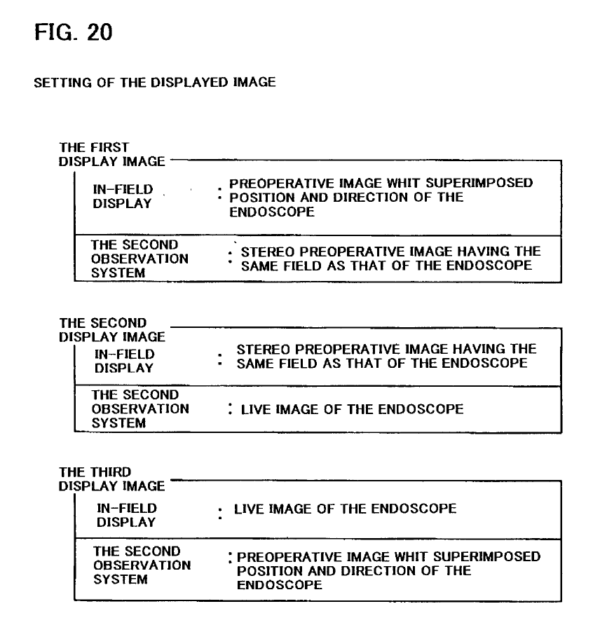

When the operator presses the setting mode button, the image control section 46 receives a signal and displays a display image setting screen as shown in FIG. 20 on a monitor 185. The display image setting screen displays description of images displayed by each of the in-field display section and the second observation optical system in each of three display modes (a first display image, a second display image, and a third display image) that are sequentially switched.

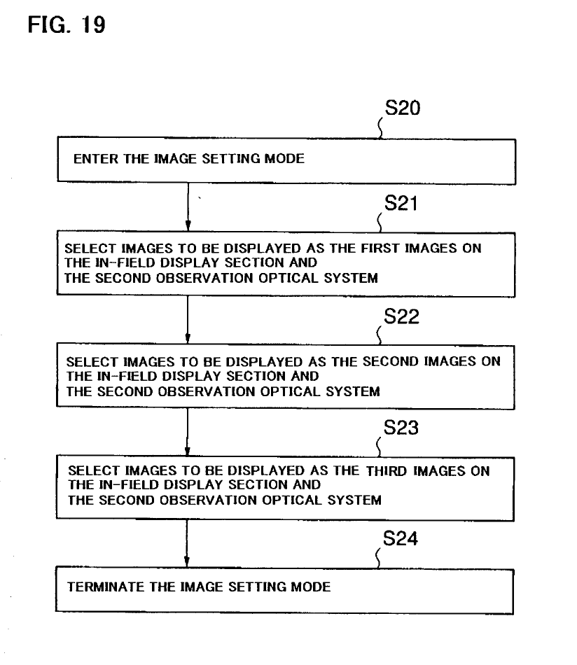

FIG. 19 is a flow chart for explaining steps of setting display screens. To set an image to be displayed in the in-field display section of the first display image, after the display image setting mode is entered (step S20), the operator selects the item of the in-field display section of the first display image is selected with upward and downward directional buttons among the switch 52 of the operating section 51. Since the display color of the item is changed when the item is selected, the operator can check the item that is being currently set. The operator selects a desired image by pressing left and right buttons of the switch 52 (step S21).

When the left and right buttons of the switch 52 are pressed, the item of the image displayed in the in-field display section of the first display image is switched to “superimposed display of an endoscope position in a preoperative image and the viewing direction of the endoscope”, followed by “display of a stereoscopic preoperative image having the same range as the observing range of the endoscope”, “display of an image observed by the endoscope”, and then “no display”. The operator stops selecting with the left and right switches when the desired display is obtained. Further, the operator presses the upward and downward buttons of the switch 52 to select the item of the second observation optical system of the first display image as the next item to be set. When the setting of the second observation optical system of the first display image is completed, the operator sequentially selects a desired image as the second display image with the left and right buttons (step S22). The operator also selects a desired image as the third display image similarly (step S23). The operator then terminates the display image setting mode (step S24).

When the setting of the display image of each item is completed as described above, the operator presses the setting mode button (not shown) provided on the operating section 51. Then, the image control section 46 updates the order of display images with the order thus set. When the setting is made as shown in FIG. 20, the images displayed in the in-field display section and the second observation optical system are switched from the first display image to the second display image and from the second display image to the third display image each time the switch A 142 is pressed. When the switch B 143 is pressed, the image in the in-field display section and the image displayed by the second observation system change places with each other.

As described above, the operator can set a desired order of display. For example, an image observed by the endoscope may be displayed in the in-field display section, and nothing is displayed in the second observation optical system 36. This setting is preferable when a treatment is performed while observing an image observed by the surgical microscope and an image observed by the endoscope in the in-field display section after the position of the endoscope during the operation is determined.

Alternatively, the same image observed by the endoscope may be displayed by both of the in-field display section and the second observation optical system 36. After the position of the endoscope during an operation is determined, the operator performs the treatment while observing an image observed by the surgical microscope and an image observed by the endoscope in the in-field display section. When closer observation is needed, details are checked on an endoscope image displayed by the second observation optical system.

In the fifth embodiment of the invention, since images are simultaneously displayed by the in-field display section and the second observation optical system 36, the steps of image switching can be eliminated unlike the case wherein only the in-field display section is used. An image desired by an operator can be displayed with improved ease of observation by replacing images displayed by the in-field display section and the second observation optical system 36 with each other.

Further, since an order for display images can be set, images can be displayed according to usage desired by an operator. This makes it possible to reduce the time required for treating an operated part and to reduce fatigue of the operator and burdens to the patient consequently.

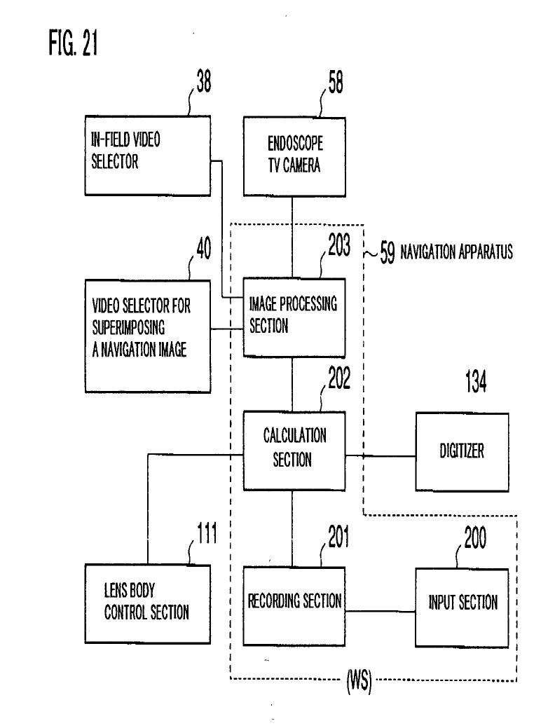

A sixth embodiment of the invention is characterized in that a part to be observed by an operator is input in advance and in that an endoscope is guided to the desired position for observation when it is inserted. The sixth embodiment will be described with reference to FIGS. 21 to 24.

As shown in FIG. 21, the sixth embodiment is characterized in that additions are made to the internal configuration of a navigation apparatus 59 according to the third embodiment.

An endoscope TV camera 58 is connected to a navigation apparatus 59. A recording section 201 in which a preoperative image is recorded and an input section 200 for inputting a desired position in the preoperative image are connected to the navigation apparatus 59. There is also connected a digitizer 134 for detecting an observing position of an endoscope and an observing position of a surgical microscope and a calculation section 202 for performing position calculations. Connected to the calculation section 202 are a lens body control section 111 for detecting information of the magnification, sight, observing position of an observation optical system of the surgical microscope and an image processing section 203 for performing image processing on an endoscope image and the preoperative image. The endoscope TV camera 58, an in-field video selector 38, and a video selector 40 for superposing a navigation image are connected to the image processing section 203.

Operations of the sixth embodiment will now be described.

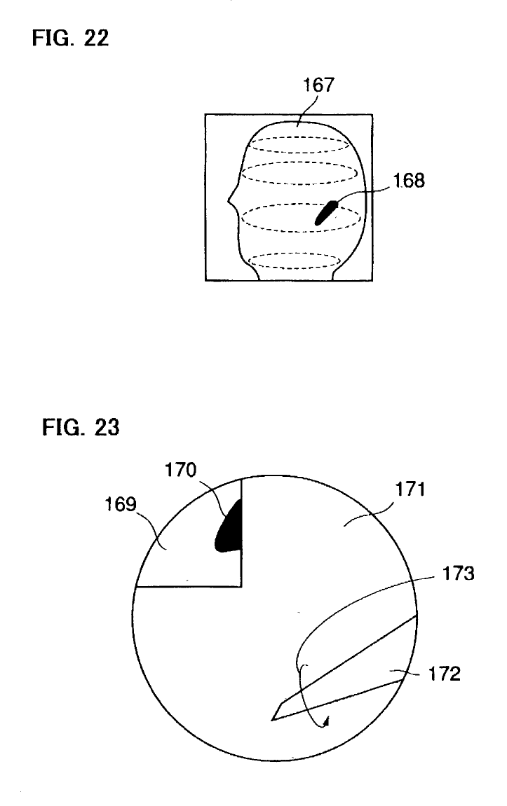

A position 168 desired by an operator and a model number of the endoscope are input in a preoperative image 167 as shown in FIG. 22 with the input section 200 in a workstation provided in the navigation apparatus 59.

The operator moves the lens body close to the operator's desired position 168 and starts observing the operated part. When the operator presses a switch A 142, an image observed by the endoscope is transmitted from the endoscope TV camera 58 to the image processing section 203. The calculation section 202 processes the observing position of the endoscope detected by the digitizer 134, information of the field of observation of the endoscope recorded in the recording section 201 (e.g., the length, viewing angle, and the aperture of the end of the endoscope), and the operator's desired position 168 in the preoperative image input from the input section 202 to calculate a positional relationship between the image observed by the endoscope and the operator's desired position 168 in the preoperative image. Based on the positional relationship, the image processing section 203 generates an observation image that is the image observed by the endoscope with a contour of the operator's desired position 168 in the preoperative image superposed thereon. This image 169 (FIG. 23) is sent to the in-field video selector 38 to display an image 170 (FIG. 23) that is the endoscope image 169 with the contour of the operator's desired position 168 superposed thereon.

The digitizer 134 also detects the observing position of the surgical microscope. Further, the calculation section 202 calculates the observing position of the surgical microscope, information of the sight and magnification of the surgical microscope transmitted from the lens body control section 111, the observing position of the endoscope, and the operator's desired position 168 input in the preoperative image. Based on the calculation, the calculation section 202 calculates the direction in which the endoscope is to be moved toward the operator's desired position 168 input in the preoperative image. The image processing section 203 generates an arrow to indicate the moving direction and transmits the same to the video selector 40 for superposing navigation image to superpose it on the image observed by the surgical microscope as an arrow 173.

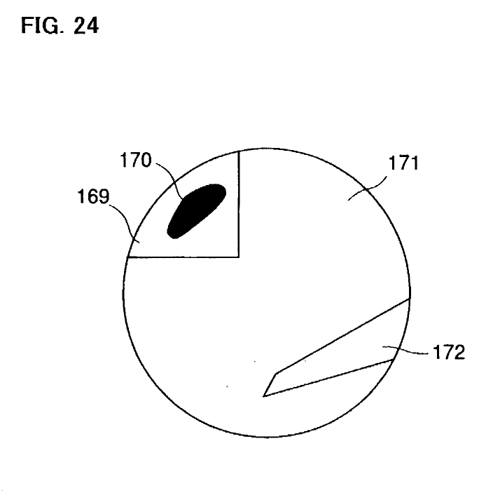

When the endoscope is rotated in the direction indicated by the arrow 173, it is guided to the image 170 in the desired position input in the preoperative image by the operator (FIG. 24).

In the present embodiment, images are switched in the in-field display section with the switch A 142. A switch B 143 has a function of turning on and off display of a contour indicating a part desired by an operator in an image displayed in the in-field display section. Specifically, when the contour is a hindrance, the switch B 143 is pressed to stop the display of the superposition of the contour by transmitting a signal from the endoscope TV camera 58 to the image processing section 203.

The sixth embodiment makes it possible to reach a part to be operated smoothly especially when the position to be observed is a part such as a bone that is less likely to be displaced from a preoperative image after craniotomy is carried out. This reduces fatigue of the operator and operating time and consequently reduces burdens to the patient.

In the seventh embodiment of the invention, images observed by an operator during an operation include an image observed by a surgical microscope, an in-field image inserted in the image observed by the surgical microscope, and an image displayed by a second observation optical system. There are two video signals, i.e., a video signal obtained by synthesizing the image observed by the surgical microscope and the in-field image inserted in the image observed by the surgical microscope and a video signal for the image displayed by the second observation optical system. To record those video signals (with VTR's for example), two VTR's may be used to record the video signals, respectively.

In this case, it is preferable to record the two video signals simultaneously during an operation. This results in a need for operating two VTR's simultaneously, which is troublesome. The present embodiment focuses on this point, and there is provided a microscopic system capable of simultaneously recording and reproducing an image observed by a surgical microscope, an image in an in-field display section, and an image displayed by a second observation optical system. The present embodiment will be described below with reference to FIGS. 25, 26, and 27.

The present embodiment has a configuration that is basically similar to the configuration of the fifth embodiment. Therefore, the description will be focused only on differences from the fifth embodiment.

In FIG. 25, some additions are made to the configuration in FIG. 15, and a configuration is shown that is aimed at recording of an image observed by a surgical microscope, an image in an in-field display section, and an image displayed by a second observation optical system 36. An image taking apparatus 173 for taking an image observed by a surgical microscope provided in a lens body of the surgical microscope is connected to a CCU 174. The CCU is connected to an image mixer 204 to which an in-field video selector 38 and a VTR control section 175 for controlling two VTR's to be described later are connected.

A video selector 39 for image observation and VTR's A 177 and B 178 as recording and reproducing apparatus are connected to the VTR control section 175. Further, a VTR operating section 176 for operating the VTR control section 175 is connected to the VTR control section 175.

An image processing apparatus 179 for performing image processing for displaying a plurality of images on a monitor 180 is connected to the VTR's A 177 and B 178.

FIG. 27 shows a configuration in which a W-VHS recorder 184 is used instead of using two VTR's as in FIG. 25.

Operations of the seventh embodiment will now be described.

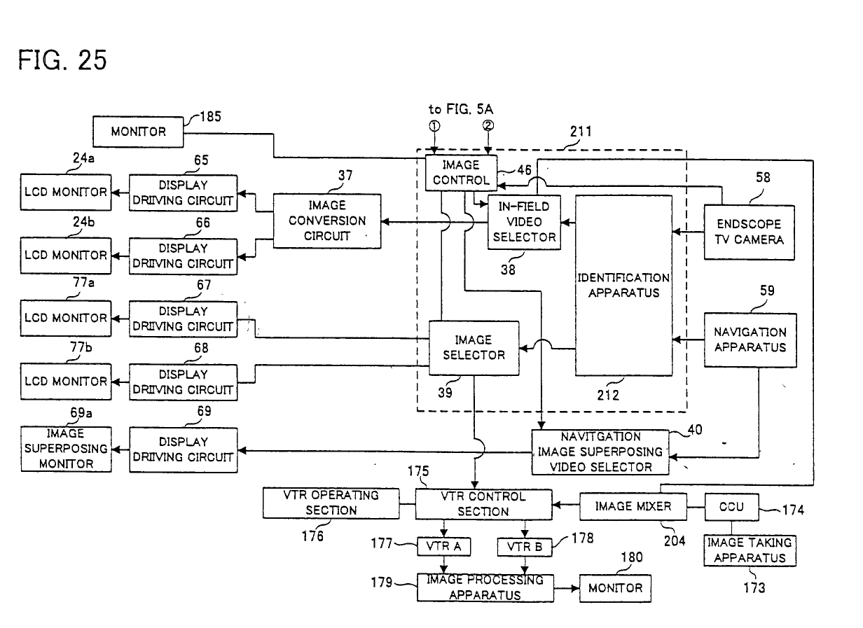

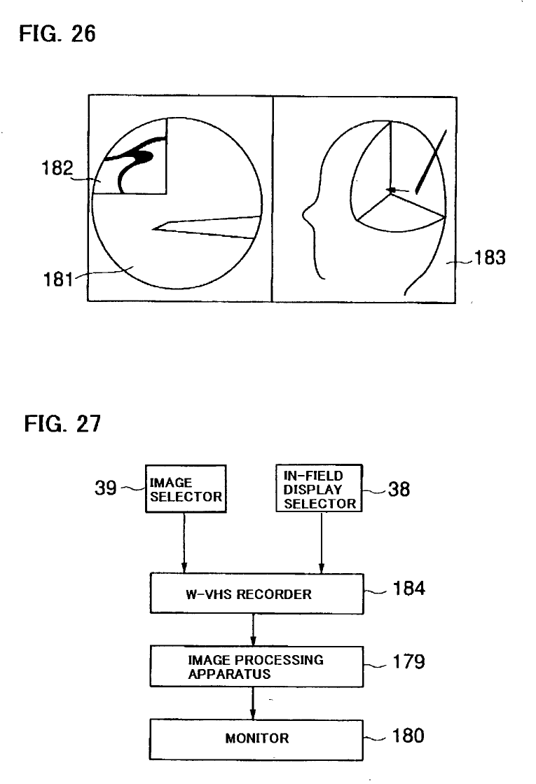

For example, an operator conducts observation from an image from the surgical microscope, an image displayed in the in-field display section, and an image from the second observation optical system 36, as shown in FIG. 16C. The image mixer 204 synthesizes the image observed by the surgical microscope and the in-field image displayed by the in-field video selector 38 into a surgical microscope image to be observed by the operator (181 in FIG. 26).

The image 181 synthesized by the image mixer 204 and an image 183 obtained by the second observation optical system 36 selected by the video selector 40 for image observation are input to the VTR control section 175. When a recording switch provided at the VTR operating section 176 is operated, the VTR control section 175 transmits a signal to cause the VTR's A 177 and B 178 to start recording simultaneously. The image 181 synthesized by the image mixer 204 is recorded by the VTR A 177, and the image (183 in FIG. 26) displayed by the second observation optical system 36 is recorded by the VTR B 178.

The recorded images are sent from the VTR's A 177 and B 178 to the image processing apparatus 179 and displayed on the monitor 180 as shown in FIG. 26.

To reproduce the recorded images, a reproduction switch SW provided at the VTR operating section 176 is operated. Then, the VTR control section 175 causes the VTR's A 177 and B 178 to start reproduction simultaneously. This reproduces the image 181 from the surgical microscope and an endoscope image 182 that have been observable for the operator during the operation simultaneously and the preoperative image 183 on which the observing direction of the endoscope is superposed, as shown in FIG. 26.

In a configuration utilizing the W-VHS recorder 184 as shown in FIG. 27, the W-VHS recorder 184 is operated to record the image from the surgical microscope, the in-field image inserted in the image from the surgical microscope, and the image obtained by the second observation optical system 36 simultaneously on a single recording tape. Referring to reproduction, the W-VHS recorder 184 is operated again to reproduce the image 181 from the surgical microscope and an endoscope image 182 that have been observable for the operator during the operation simultaneously and the preoperative image 183 on which the observing direction of the endoscope is superposed, as shown in FIG. 26.

The seventh embodiment makes it possible to record and reproduce an image observed by a surgical microscope, an image in an in-field display section, and a second observed image simultaneously. It is therefore possible to prepare a record of an operation and to indicate images that the operator observes to conduct the operation.

An eighth embodiment of the invention has a configuration that is basically similar to the configuration of the fifth embodiment. Operations of the eighth embodiment that are different from those of the fifth embodiment will be described.

A navigation apparatus 59 calculates the orientation of a preoperative image relative to a lens body 104 from information detected on the lens body 104 by a digitizer 134.

An operator presses a switch A 142 provided on an endoscope. A signal indicating the press on the switch 142 A is then sent to a display image control section 46. The display image control section 46 sends a signal to an in-field display selector 38. Upon receipt of the signal, the in-field display selector 38 transmits an image to a video conversion circuit section 37.

As a result, an image 301 is displayed in an in-field display section of an image 300 observed by a surgical microscope, as shown in FIG. 28. In this case, the image 301 in the in-field display section includes an image 302 that is a preoperative image taken in the same direction as the observing direction of the surgical microscope and superposed with the position of the endoscope inserted into the operated part. Since the image 301 and the image 300 observed by the surgical microscope are taken in the same direction, the image 302 displayed in the in-field display section and an image 303 of the endoscope displayed in the image observed by the surgical microscope are in the same direction. The operator conducts observation while comparing the position of the endoscope inserted in the operated part with the image observed by the surgical microscope on the preoperative image.

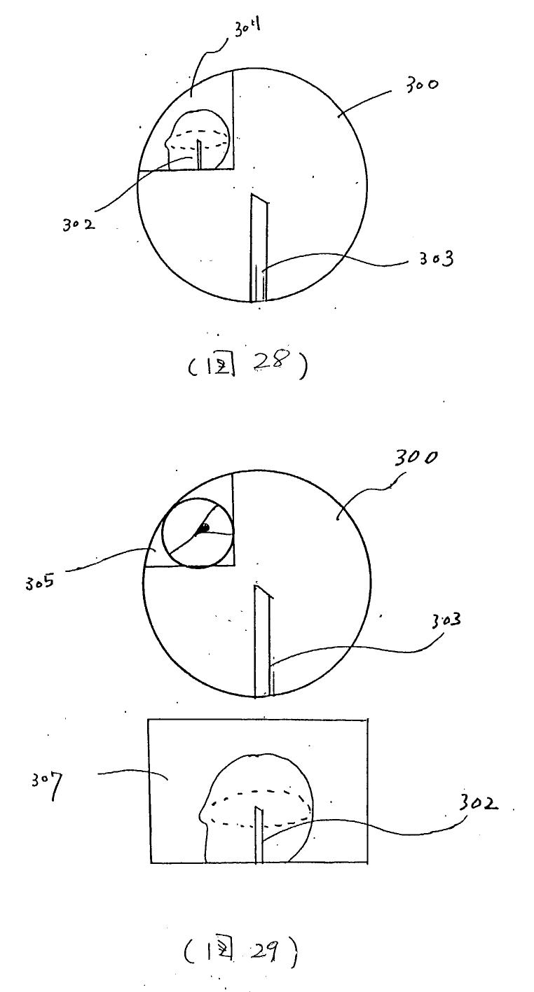

When the switch 142 A is pressed next, as shown in FIG. 29, the display control section 46 switches the image displayed in the in-field display section. A stereoscopic preoperative image 305 within the observation range of the endoscope is displayed in the in-field display section. From this image, a preoperative image of the part to be checked with the endoscope is examined in comparison to the actual position of insertion. The image 301 displayed in the in-field display section in FIG. 28 is displayed in an enlarged scale as an image 307 from the second optical system.

The operator then presses the switch 142 A further. As shown in FIG. 30, an image 310 observed by the endoscope is displayed in the in-field display section. The second observation optical system displays a preoperative image 312 in the same direction as the observing direction of the surgical microscope, and a preoperative image 313 of the part to be observed with the endoscope is displayed in a top left part of the same (the images are synthesized with an image mixer that is not shown). The images allow the operator to observe an endoscope image in the desired position. The operator observes the actual observed image in comparison to the preoperative image having the same range by moving his or her line-of-sight to the greater screen.

The present embodiment allows an operator to observe all of an image observed by a surgical microscope, an image observed by an endoscope, a preoperative image in the same direction as the observing direction of the surgical microscope, and a preoperative image in the same direction as the observing direction of the endoscope with an understanding of correlation between them. This makes it possible to observe a desired position quickly even when the observation is conducted with the endoscope at a dead angle to the surgical microscope.

Obviously, the same advantages can be achieved by using an ultrasonic probe instead of an endoscope. Examples of images obtained in such a case are described below.

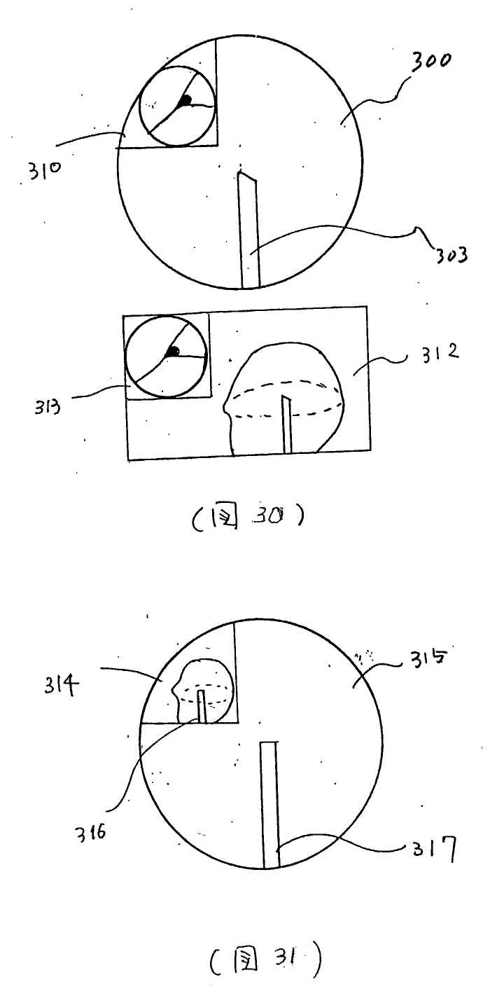

Referring to FIG. 31, an image 314 is displayed in an in-field display section of an image 315 observed by a surgical microscope. The preoperative image 314 is a preoperative image taken in the same direction as the observing direction of the surgical microscope on which an image of an ultrasonic probe 316 is superposed. The same ultrasonic probe is indicated by reference numeral 317 also in the image 315 observed by the surgical microscope.

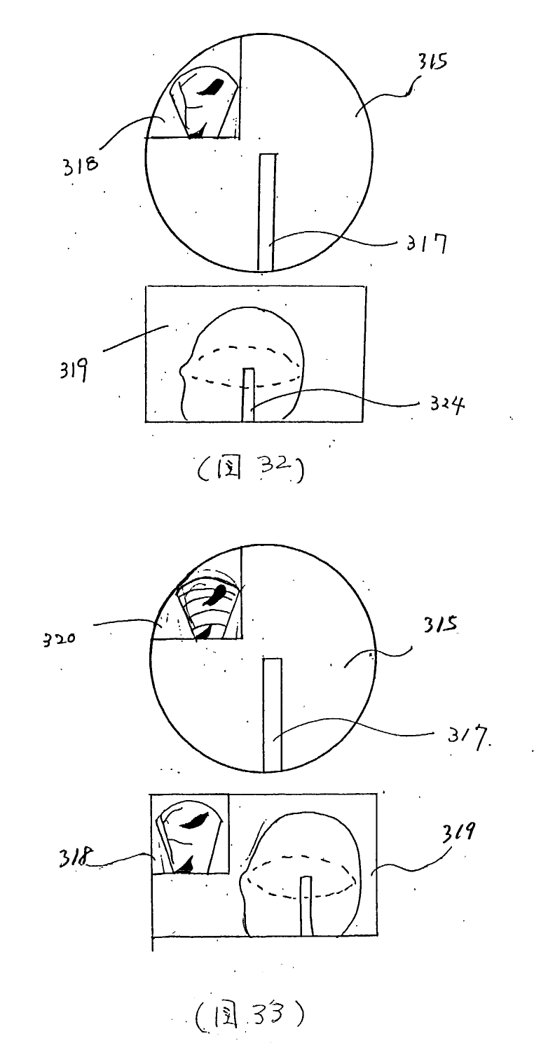

Referring to FIG. 32, an image displayed in an in-field display section is a preoperative image 318 taken in the same direction as the observing direction of a surgical microscope, the image showing a region having the same range as a range that is observed with an ultrasonic probe. An image 319 displayed by a second observation optical system is an image similar to the image 314 displayed in the in-field display section in FIG. 31. An image of the ultrasonic probe is superposed on the image 319 with reference numeral 324.

Referring to FIG. 33, an ultrasonically observed image 320 is displayed in an in-field display section. Images similar to the image 318 in the in-field display section in FIG. 32 and the image 319 in the second observation optical system in FIG. 32 are allocated to a second observation optical system.

Thus, each of the above embodiments of the invention makes it possible to recognize the position of an inserted endoscope (or ultrasonic observation apparatus) easily and to make orientation of the endoscope (or ultrasonic observation apparatus) in an image observed by a surgical microscope easy to recognize.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be construed to cover all modifications that may fall within the scope of the appended claims.

What is claimed is:

1. A microscopic system comprising:
a microscope section for stereoscopically observing a part under operation;
a subsection for observing the part under operation;
an image display section capable of displaying a plurality of images; and
a display control section for selectively displaying at least one of the plurality of images simultaneously with an image observed by the microscope section.

2. The microscopic system of claim 1, wherein the plurality of images is selected from a group consisting of a preoperative image having a wide range including the vicinity of the part under operation, a preoperative image having the same range as an observing range of the subsection for observation, and an image observed by the subsection for observation.

3. A microscopic system according to claim 1, wherein the display control section comprises:
an input section for switching the plurality of images displayed in the image display section; and
an image switching section for sequentially switching the images displayed in the image display section according to an input from the input section.

4. A microscopic system according to claim 1, wherein the display control section comprises an image synthesizing section for synthesizing the plurality of images displayed in the image display section in a predetermined combination.

5. A microscopic system according to claim 2, wherein the preoperative image having a wide range including the vicinity of the part under operation is an image that is correlated with an observing position and an observing direction of the microscope.

6. A microscopic system according to claim 1, wherein the subsection for observation is an endoscope.

7. A microscopic system according to claim 1, wherein the subsection for observation is an ultrasonic observation apparatus.

8. A microscopic system according to claim 3, wherein the input section comprises a plurality of switches.

9. A microscopic system according to claim 8, wherein the display control section has a control section for controlling the order of switching of the images at the image switching section according to states of input of the plurality of switches.

10. A microscopic system comprising:
a microscope section for magnified observation of a part under operation;
an endoscope for observing the part under operation;
an image display section capable of displaying a plurality of images;
a switching section for switching the plurality of images displayed in the image display section; and
a display control section for controlling an order of switching of the images displayed in the image display section according to an instruction for switching from the switching section.

11. The microscopic system of claim 10, wherein the plurality of images is selected from a group consisting of a preoperative image having a wide range including a vicinity of the part under operation, a stereoscopic preoperative image having the same range as an observing range of the endoscope, and an image observed by the endoscope.

12. A microscopic system according to claim 10, wherein the image display section has at least one of:
an in-field display section for displaying an electronic image in the field of view of the microscope section;
a second observation optical system for displaying an electronic image; and
an image superposing section for performing superposed display in a field of observation of the microscope.

13. A microscopic system according to claim 10, wherein the switching section has two or more input sections.

14. A microscopic system according to claim 10, wherein the display control section has an identification section for identifying an input image.

15. A microscopic system according to claim 14, wherein the identification section has a transmission signal identifying section for identifying a signal transmitted to the display control section.

16. A microscopic system according to claim 14, wherein the identification section has a connecting section identifying section for identifying a connecting section connected to the image control section.

17. A microscopic system according to claim 10, wherein the display control section has a setting change section for making a change to provide a desired order of display images.

18. A microscopic system according to claim 12, comprising:
a recording section for recording and reproducing an image observed by the microscopic section and images in the in-field display section and the second observation optical system; and
a recording section control section for controlling the recording section for recoding and reproducing the image observed by the microscopic section and the images in the in-field display section and the second observation optical system simultaneously.

19. A microscopic system comprising:
a microscope section for magnified observation of a part under operation;
an superimposed display section for performing superimposed display of an electronic image in a field of view of the microscope section;
an endoscope for observing the part under operation;
an observing position detecting section for detecting observing positions of the microscope section and the endoscope;
an input section for setting a desired position in a medical image;
a calculation section for identifying positional correlation between the observing position detecting section and the desired position input by the input section; and
an image processing section for generating an image to be superposed at the superposed display section based on correlated position information obtained by the observing position detecting section or the calculation section.

20. The microscopic system of claim 19, wherein the medical image is a preoperative image.

21. A medical observation apparatus comprising;
a scope for observing a part under operation;
a memory for storing preoperative data;
a navigation section for generating a preoperative image having substantially the same range as that of an image observed by the scope by using the preoperative data recorded in the memory;
a selector for selectively outputting at least the image observed by the scope or the preoperative image generated by the navigation section; and
a display for displaying the image output by the selector.

22. A medical observation apparatus according to claim 21, further comprising a section for detecting the position of the scope, wherein the navigation section generates the preoperative image utilizing the detected position of the scope.

23. A medical observation apparatus according to claim 21, wherein the image selected by the selector is switched according to an instruction from an operator.

24. A method of displaying a medical image for displaying an image of a part under operation, the method comprising:
outputting an image observed by a scope as a first video signal;
generating a preoperative image having substantially the same range as an observing range of the scope using preoperative data recorded in a memory and outputting the preoperative image same as a second video signal;
selecting one signal among signals including the first video signal and the second video signal with a selector based on an instruction from an operator; and
displaying the video signal selected with the selector on a display.

25. A method of displaying a medical image for displaying an image of a part under operation, the method comprising:
outputting an image observed by a scope as a first video signal;
generating a preoperative image having substantially the same range as an observing range of the scope using preoperative data recorded in a memory and outputting the preoperative image as a second video signal;
generating a preoperative image having a range wider than the observing range of the scope using the preoperative data recorded in the memory and outputting the preoperative image as a third video signal;
selecting one signal among signals including the first video signal, second video signal, and third video signal with a selector based on an instruction from an operator; and
displaying the video signal selected with the selector on a display.

26. A method of displaying a medical image for displaying an image of a part under operation according to claim 25, wherein the selector selects a signal according to the position or type of a connector to which the video signal is input.

27. A method of displaying a medical image for displaying an image of a part under operation according to claim 25, wherein the selector selects a signal according to an identification signal that identifies the video signal.
